(19) United States
(12) Patent Application Publication
(10) Pub. No.: US 2011/0152326 A1
(43) Pub. Date: Jun. 23, 2011

(54) SUBSTITUTED ARYLOXOETHYL CYCLOPROPANECARBOXAMIDE COMPOUNDS AS VR1 RECEPTOR ANTAGONISTS

(76) Inventors: Takeshi Hanazawa, Aichi-ken (JP); Satoshi Nagayama, Aichi-ken (JP); Kazumari Nakao, Aichi-ken (JP); Hirotaka Tanaka, Aichi-ken (JP)

(21) Appl. No.: 11/909,843

(22) PCT Filed: Mar. 15, 2006
(86) PCT No.: PCT/IB2006/000585

§ 371 Date: Mar. 4, 2011

Related U.S. Application Data
(60) Provisional application No. 60/665,937, filed on Mar. 28, 2005.

(51) Int. Cl. A61K 31/44; C07D 211/70; C07C 233/61; C07D 233/22; A61K 31/165; A61K 31/4164; A61P 9/10; A61P 25/00; A61P 29/00; A61P 25/06; A61P 13/10; A61P 25/28; A61P 11/00; A61P 11/06; A61P 1/00; A61P 3/04

(52) U.S. Cl. 514/354; 546/328; 564/161; 548/333.5; 514/617; 514/400

(57) ABSTRACT

This invention provides a compound of the formula (I): (I) These compounds are useful for the treatment of disease conditions caused by overactivation of the VR1 receptor, such as pain, or the like in mammalian. This invention also provides a pharmaceutical composition comprising the above compound.

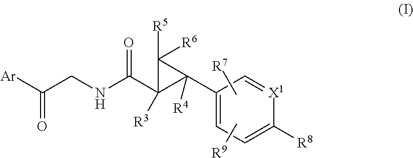

### INTRODUCTION

This invention relates to novel substituted aryl and heteroaryl oxoethyl cyclopropanecarboxamide compounds. These compounds are useful as antagonists of the Type I Vanilloid Receptor (VR1), and are thus useful for the treatment of pain, neuralgia, neuropathies, nerve injury, burns, migraine, carpal tunnel syndrome, fibromyalgia, neuritis, sciatica, pelvic hypersensitivity, bladder disease, inflammation, or the like in mammals, especially humans. The present invention also relates to a pharmaceutical composition comprising the above compounds.

The Type I Vanilloid Receptor (VR1) is a ligand gated non-selective cation channel. It is believed to be a member of the transient receptor potential super family. VR1 is recognized as a polymodal nociceptor that integrates multiple pain stimuli, e.g., noxious heat, protons, and vanilloids (European Journal of Physiology 451:151-159, 2005). A major distribution of VR1 is in the sensory (Aδ- and C-) fibers, which are bipolar neurons having somata in sensory ganglia. The peripheral fibers of these neurons innervate the skin, the mucosal membranes, and almost all internal organs. It is also recognized that VR1 exists in bladder, kidney, brain, pancreas, and various kinds of organs. A body of studies using VR1 agonists, e.g. capsaicin or resiniferatoxin, have suggested that VR1 positive nerves are thought to participate in a variety of physiological responses, including nociception (Clinical Therapeutics. 13(3): 338-395, 1991, Journal of Pharmacology and Experimental Therapeutics 314:410-421, 2005, and Neuroscience Letter 388: 75-80, 2005). Based on both the tissue distribution and the roles of VR1, VR1 antagonists would have good therapeutic potentials.

International Patent Application Number WO-A-200216318 discloses a variety of sulfonylaminobenzylthiourea derivatives and N-sulfonylaminobenzy-2-phenoxyacetamide derivatives as modulators for the vanilloid receptor.

International Patent Application Number WO-A-2004047738 discloses a variety of arylcyclopropylcarboxylic amides as potassium openers.

It is desirable to provide VR1 antagonists improved properties such as potent binding activity with the VR1 receptor by systemic administration. Other potential advantages include less toxicity, good absorption, good half-life, good solubility, low protein binding affinity, less drug-drug interaction, a reduced inhibitory activity at HERG channel, reduced QT prolongation and good metabolic stability.

### BRIEF DISCLOSURE OF THE INVENTION

It has now been found that substituted aryl and heteroaryl oxoethyl cyclopropanecarboxamide compounds are VR1 antagonists with analgesic activity by systemic administration.

The present invention provides a compound of the following formula (I):

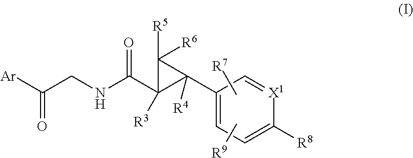

wherein Ar represents

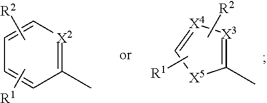

X¹ represents CH, CR⁷ or N;
X² represents CH, CR¹ or N;
X³ represents N, X⁴ represents CH or CR¹ and X⁵ represents S, NH or NR²; or X³ represents CH or CR¹,
X⁴ represents N and X⁵ represents NH or NR²;
R¹, R², R⁷ and R⁹ each independently represent hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl, (C₁-C₆)alkylsulfonyl, [(C₁-C₆)alkyl]NH—, [(C₁-C₆)alkyl]₂N—, H₂N—(C₁-C₆)alkoxy, (C₁-C₆)alkyl-NH—(C₁-C₆)alkoxy, [(C₁-C₆)alkyl]₂N(C₁-C₆)alkoxy; H₂N—(C₁-C₆)alkoxy-(C₁-C₆)alkyl, (C₁-C₆)alkyl-NH—(C₁-C₆)alkoxy-(C₁-C₆)alkyl, or [(C₁-C₆)alkyl]₂N(C₁-C₆)alkoxy-(C₁-C₆)alkyl;
R³, R⁴, R⁵ and R⁶ each independently represent hydrogen, halogen, (C₁-C₆)alkyl, hydroxy(C₁-C₆)alkyl or halo(C₁-C₆)alkyl; and
R⁸ represents halogen, (C₁-C₆)alkyl, halo(C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, [(C₁-C₆)alkyl]NH—, or [(C₁-C₆)alkyl]₂N—, or R⁷ and R⁸, when attached to adjacent carbon atoms, may be taken together with the carbon atoms to which they are attached to form a 5- to 8-membered cycloalkyl ring or heterocyclic ring in which one or two non-adjacent carbon atoms are optionally replaced by oxygen, sulfur or NH groups, wherein the cycloalkyl ring or the heterocyclic ring is unsubstituted or substituted with one or more substituents selected from the group consisting of hydroxy, (C₁-C₆)alkyl, (C₁-C₆)alkoxy and hydroxy(C₁-C₆)alkyl;
or a pharmaceutically acceptable salt or solvate thereof.

### DETAILED DESCRIPTION OF THE INVENTION

As used herein, the term “halogen” means fluoro, chloro, bromo and iodo, preferably fluoro and chloro.

As used herein, the term “aryl” means a monocyclic or bicyclic aromatic carbocyclic ring of 6 to 10 carbon atoms; or bicyclic partially saturated carbocyclic ring of 6 to 10 carbon atoms including, but not limited to, phenyl, naphthyl, indanyl, indenyl and tetralinyl. Preferred aryl groups are phenyl, indanyl and naphthyl.

As used herein, the term “(C₁-C₆)alkyl” means straight or branched chain saturated radicals having from one to six carbon atoms, including, but not limited to, methyl, ethyl, n-propyl, iso-propyl, n-butyl, iso-butyl, secondary-butyl and tertiary-butyl. Preferred (C₁-C₆)alkyl groups are methyl, ethyl, n-propyl, n-butyl and tertiary-butyl.

As used herein, the term “hydroxy(C₁-C₆)alkyl” means an (C₁-C₆)alkyl radical as defined above which is substituted by a hydroxy group including, but not limited to, hydroxymethyl, hydroxyethyl, hydroxy n-propyl, hydroxyisopropyl, hydroxy n-butyl, hydroxy iso-butyl, hydroxy secondary-butyl and hydroxy tertiary-butyl. Preferred hydroxyalkyl groups are hydroxymethyl, hydroxyethyl, hydroxy n-propyl and hydroxy n-butyl.

As used herein, the term “(C₁-C₆)alkoxy” means (C₁-C₆)alkyl-O—, including, but not limited to, methoxy, ethoxy, n-propoxy, iso-propoxy, n-butoxy, iso-butoxy, secondary-butoxy and tertiary-butoxy. Preferred alkoxy groups are methoxy, ethoxy, n-propoxy, n-butoxy and tertiary-butoxy.

As used herein, the term “hydroxy(C₁-C₆)alkoxy” means a (C₁-C₆)alkoxy radical as defined above which is substituted by a hydroxy group including, but not limited to, hydroxymethoxy, hydroxyethoxy, hydroxy n-propoxy, hydroxyisopropoxy, hydroxy n-butoxy, hydroxy iso-butoxy, hydroxy secondary-butoxy and hydroxy tertiary-butoxy. Preferred hydroxyalkoxy groups are hydroxymethoxy, hydroxyethoxy, hydroxy n-propoxy and hydroxy n-butoxy.

As used herein, the term “(C₁-C₆)alkylthio” means (C₁-C₆)alkyl-S— wherein (C₁-C₆)alkyl is defined above, including, but not limited to, methylthio, ethylthio, n-propylthio, iso-propylthio, n-butylthio, iso-butylthio, secondary-butylthio and tertiary-butylthio. Preferred alkylthio groups are methylthio, ethylthio, n-propylthio and n-butylthio.

As used herein, the term “(C₁-C₆)alkylsulfinyl” means (C₁-C₆)alkyl-SO— wherein (C₁-C₆)alkyl is defined above, including, but not limited to, methylsulfinyl, ethylsulfinyl, n-propylsulfinyl, iso-propylsulfinyl, n-butylsulfinyl, iso-butylsulfinyl, secondary-butylsulfinyl and tertiary-butylsulfinyl. Preferred alkylsulfinyl groups are methylsulfinyl, ethylsulfinyl, n-propylsulfinyl and n-butylsulfinyl.

As used herein, the term “(C₁-C₆)alkylsulfonyl” means (C₁-C₆)alkyl-SO₂— wherein (C₁-C₆)alkyl is defined above, including, but not limited to, methylsulfonyl, ethylsulfonyl, n-propylsulfonyl, iso-propylsulfonyl, n-butylsulfonyl, iso-butylsulfonyl, secondary-butylsulfonyl and tertiary-butylsulfonyl. Preferred alkylsulfonyl groups are methylsulfonyl, ethylsulfonyl, n-propylsulfonyl and n-butylsulfonyl.

As used herein, the term “[(C₁-C₆)alkyl]NH-” means (C₁-C₆)alkyl-NH— wherein (C₁-C₆)alkyl is defined above, including, but not limited to, methylamino, ethylamino, n-propylamino, iso-propylamino, n-butylamino, iso-butylamino, secondary-butylamino and tertiary-butylamino. Preferred alkylamino groups are methylamino, ethylamino, n-propylamino and n-butylamino.

As used herein, the term “[(C₁-C₆)alkyl]₂N-” means di[(C₁-C₆)alkyl]-N— wherein (C₁-C₆)alkyl is defined above, including, but not limited to, dimethylamino, diethylamino, methylethylamino, di n-propylamino, methyl n-propylamino, ethyl n-propylamino di iso-propylamino, di n-butylamino, methyl n-butylamino di iso-butylamino, di secondary-butylamino and di tertiary-butylamino. Preferred dialkylamino groups are dimethylamino, diethylamino, di n-propylamino and di n-butylamino.

As used herein the term “halo(C₁-C₆)alkyl”, means a (C₁-C₆)alkyl radical which is substituted by one or more halogen atoms as defined above including, but not limited to, fluoromethyl, difluoromethyl, trifluoromethyl, 2-fluoroethyl, 2,2-difluoroethyl, 2,2,2-trifluoroethyl, 2,2,2-trichloroethyl, 3-fluoropropyl, 4-fluorobutyl, chloromethyl, trichloromethyl, iodomethyl and bromomethyl. Preferred haloalkyl groups are fluoromethyl, difluoromethyl, trifluoromethyl, 2-fluoroethyl, 2,2-difluoroethyl and 2,2,2-trifluoroethyl,

As used herein, the term “cycloalkyl ring” means a saturated carbocyclic ring of from 3 to 8 carbon atoms including, but not limited to, cyclopropyl, cyclobutyl, cyclopentyl, cyclohexyl, cycloheptyl and cyclooctyl. Preferred cyclic rings are cyclopentyl and cyclohexyl. The cycloalkyl ring is optionally substituted with one or more substituents selected from the group consisting of hydroxy, (C₁-C₆)alkyl, (C₁-C₆)alkoxy and hydroxy(C₁-C₆)alkyl.

As used herein the term “heterocyclic ring” means a 3- to 8-membered cycloalkyl ring in which one or two non-adjacent carbon atoms are optionally replaced by oxygen, sulfur or NH group. Examples of such heterocyclic rings include, but are not limited to, tetrahydrofuran, tetrahydrothiophen, tetrahydrothiazole, tetrahydropyrrole, tetrahydropyran, tetrahydropyridine, tetrahydroprazine, and tetrahydropyrimidine. Preferred heterocyclic rings are tetrahydrofuran, tetrahydrothiophen, tetrahydropyrrole and tetrahydropyridine. The heterocyclic ring is optionally substituted with one or more substituents selected from the group consisting of hydroxy, (C₁-C₆)alkyl, (C₁-C₆)alkoxy and hydroxy(C₁-C₆)alkyl.

Where the compounds of formula (I) contain hydroxy groups, they may form esters. Examples of such esters include esters with a carboxy group. The ester residue may be an ordinary protecting group or a protecting group which can be cleaved in vivo by a biological method such as hydrolysis.

Preferably Ar represents

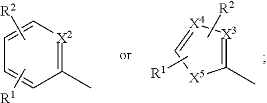

X² represents N, CH or CR¹;
X³ represents N, X⁴ represents CH, and X⁵ represents NH or NR¹ respectively; and X¹, R¹, R², R³, R⁴, R⁵, R⁶, R⁷, R⁸ and R⁹ are each as defined above.

Preferably X¹ represents CH or CR⁷; Ar is either as defined above in its broadest definition or in its preferred definition, and R¹, R², R³, R⁴, R⁵, R⁶, R⁷, R⁸ and R⁹ are each as defined above.

Preferably, R¹ and R² are each independently hydrogen, hydroxy, (C₁-C₆)alkyl, halo(C₁-C₆)alkyl and (C₁-C₆)alkoxy; Ar and X¹ are each as defined above, either in the broadest definition or the preferred definition; and R³, R⁴, R⁵, R⁶, R⁷, R⁸ and R⁹ are each as defined above; more preferably, R¹ and R² are each independently hydrogen, hydroxy, methyl, ethyl, methoxy or trifluoromethyl.

Preferably R³, R⁴, R⁵ and R⁶ are each independently hydrogen, halogen or (C₁-C₆)alkyl; Ar, X¹ and R¹ and R² are each as defined above, either in the broadest definition or the preferred definition; and R⁷, R⁸ and R⁹ are each as defined above; more preferably R³, R⁴, R⁵ and R⁶ are each independently hydrogen, fluoro or methyl.

Preferably R⁷ and R⁹ are each independently hydrogen or halogen; Ar, X¹, R¹, R², R³, R⁴, R⁵ and R⁶ are each as defined above, either in the broadest definition or the preferred definition; and R⁸ and R⁹ is as defined above; more preferably R⁷ and R⁹ are each independently hydrogen, fluoro or chloro.

Preferably R⁸ is (C₁-C₆)alkyl or halo(C₁-C₆)alkyl; and Ar, X¹, R¹, R², R³, R⁴, R⁵, R⁶, R⁷ and R⁹ are each as defined above, either in the broadest definition or the preferred definition; more preferably R⁸ is tert-butyl, trifluoromethyl or 2,2,2-trifluoro-1,1-dimethyl-ethyl.

Preferred compounds of the invention include those in which each variable in Formula (I) is selected from the preferred groups for each variable.

A preferred individual compound of this invention is selected from the compounds of the Examples, or a pharmaceutically acceptable salt or solvate thereof.

The compounds of the present invention are antagonists of the VR1 receptor and are thus useful in therapeutics, particularly for the treatment of acute cerebral ischemia, pain, chronic pain, neuropathic pain, inflammatory pain, post herpetic neuralgia, neuropathies, neuralgia, diabetic neuropathy, HIV-related neuropathy, nerve injury, rheumatoid arthritic pain, osteoarthritic pain, burns, back pain, visceral pain, cancer pain, dental pain, headache, migraine, carpal tunnel syndrome, fibromyalgia, neuritis, sciatica, pelvic hypersensitivity, pelvic pain, menstrual pain; bladder disease, such as incontinence, micturition disorder, renal colic and cystitis; inflammation, such as burns, rheumatoid arthritis and osteoarthritis; neurodegenerative disease, such as stroke, post stroke pain and multiple sclerosis; pulmonary disease, such as asthma, cough, chronic obstructive pulmonary disease (COPD) and broncho constriction; gastrointestinal, such as gastroesophageal reflux disease (GERD), dysphagia, ulcer, irritable bowel syndrome (IBS), inflammatory bowel disease (IBD), colitis and Crohn's disease; ischemia, such as cerebrovascular ischemia; emesis, such as cancer chemotherapy-induced emesis, and obesity, or the like in mammals, especially humans.

The compounds of formula (I), being VR1 receptor antagonists, are potentially useful in the treatment of a range of disorders. The treatment of pain, particularly neuropathic pain, is a preferred use.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a heightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; and
orofacial pain, including dental pain, otic pain, burning mouth syndrome and temporomandibular myofascial pain.

The present invention provides a pharmaceutical composition including a compound of formula (I), or a pharmaceutically acceptable salt or solvate thereof, together with a pharmaceutically acceptable excipient. The composition is preferably useful for the treatment of the disease conditions defined above.

The present invention further provides a compound of formula (I), or a pharmaceutically acceptable salt or solvate thereof, for use as a medicament.

Further, the present invention provides a method for the treatment of the disease conditions defined above in a mammal, preferably a human, which includes administering to said mammal a therapeutically effective amount of a compound of formula (I), or a pharmaceutically acceptable salt or solvate thereof.

Yet further, the present invention provides the use of a compound of formula (I), or a pharmaceutically acceptable salt or solvate thereof, in the manufacture of a medicament for the treatment of the disease conditions defined above.

Yet further, the present invention provides a combination of a compound of the formula (I), or a pharmaceutically acceptable salt or solvate thereof, and another pharmacologically active agent.

### General Synthesis

The compounds of the present invention may be prepared by a variety of processes well known for the preparation of compounds of this type, for example as shown in the following reaction Schemes. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1999);

The following reaction schemes illustrate the preparation of compounds of formula (I). According to a first process, compounds of formula (I) may be prepared from compounds of formula (II) as illustrated by Scheme 1.

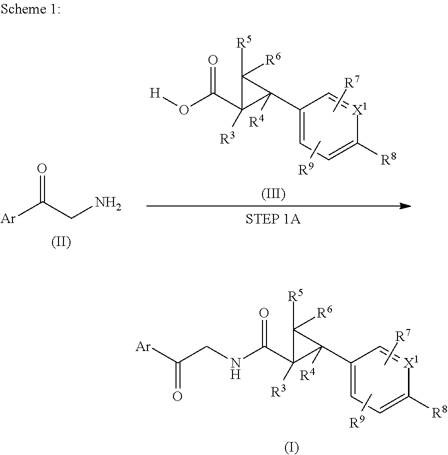

Step 1A: In this Step, a compound of formula (I) can be prepared by the coupling reaction of an amine compound of formula (II) with an acid compound of formula (III) in the presence or absence of a coupling reagent in an inert solvent.

Suitable coupling reagents are those typically used in peptide synthesis including, for example, diimides (e.g., dicyclohexylcarbodiimide (DCC) and 1-ethyl-3-(3′dimethylaminopropyl)-carbodiimide hydrochloride (EDC)), 2-ethoxy-N-ethoxycarbonyl-1,2-dihydroquinoline, 2-bromo-1-ethylpyridinium tetrafluoroborate (BEP), 2-chloro-1,3-dimethylimidazolinium chloride (CDI), benzotriazol-1-yloxy-tris(dimethylamino)phosphonium hexafluorophosphate (BOP), diethyl azodicarboxylate-triphenylphosphine, diethylcyanophosphate, diethylphosphorylazide, 2-chloro-1-methylpyridinium iodide, N,N′-carbonyldiimidazole, benzotriazole-1-yl diethyl phosphate, ethyl chloroformate or isobutyl chloroformate.

The reaction can be carried out in the presence of a base such as, 1-hydroxybenzotriazole (HOBt), N,N-diisopropylethylamine, N-methylmorpholine and triethylamine. The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: acetone; nitromethane; N,N-dimethylformamide (DMF); N-methyl-2-pyrrolidone (NMP); sulfolane; dimethyl sulfoxide (DMSO); 2-butanone; acetonitrile; halogenated hydrocarbons, such as dichloromethane, dichloroethane, chloroform; and ethers, such as tetrahydrofuran and 1,4-dioxane.

The reaction can take place over a wide range of temperatures, and the precise reaction temperature is not critical to the invention. The preferred reaction temperature will depend upon such factors as the nature of the solvent, and the starting material or reagent used. However, in general, it is convenient to carry out the reaction at a temperature of from −20 to 100° C., more preferably from about 0 to 60° C. The time required for the reaction can also vary widely, depending on many factors, notably the reaction temperature and the nature of the reagents and solvent employed. However, provided that the reaction is effected under the preferred conditions outlined above, a period of from 5 minutes to 1 week, more preferably from 30 minutes to 24 hours, will usually suffice.

Alternatively, the compound of formula (III) may first be converted to an acylhalide by the reaction with halogenating agents such as oxalylchloride, phosphorus oxychloride and thionyl chloride. The resulting acylhalide may then be coupled with a compound of formula (II) as described above.

According to a second process, when Ar is

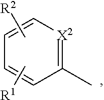

compounds of formula (II) may be prepared from compounds of formula (V) as illustrated by

Scheme 2.

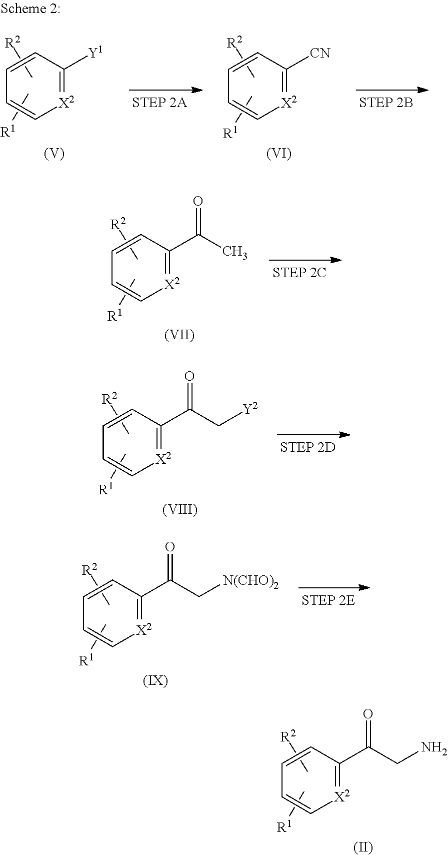

wherein Y¹ and Y² represent suitable leaving groups such as a sulfoxy group or halogen atom, for example chlorine.

Step 2A: In this step, a compound of formula (VI) can be prepared by cyanating a compound of formula (V) in the presence of a transition metal catalyst and metal cyanide reagent in an inert solvent.

Examples of suitable solvents include: tetrahydrofuran; 1,4-dioxane; N,N-dimethylformamide; acetonitrile; alcohols, such as methanol or ethanol; halogenated hydrocarbons, such as dichloromethane, 1,2-dichloroethane, chloroform or carbon tetrachloride; and dimethoxyethane. Suitable metal cyanide reagents include, for example: alkalimetal cyanide such as lithium cyanide, sodium cyanide and potassium cyanide; transition metal cyanide such as ferric(II) cyanide, cobalt(II) cyanide, copper(I) cyanide, copper(II) cyanide and ainc(II) cyanide; sodium borohydride cyanide; and trimethylsilyl cyanide.

This reaction can be carried out in the presence of a suitable catalyst. There is likewise no particular restriction on the nature of the catalyst used, and any catalyst commonly used in reactions of this type can equally be used here. Examples of such catalysts include tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, copper(II) trifluoromethanesulfonate, palladium(II) acetate, palladium(II) chloride, bisacetonitriledichloropalladium(0), bis(dibenzylideneacetone)palladium(0), tris(dibenzylideneacetone)dipalladium(0) and [1,1′-bis(diphenylphosphino)ferrocene]palladium(II) dichloride. Preferred catalysts are tetrakis(triphenylphosphine)-palladium, bis(triphenylphosphine)palladium(II) chloride, palladium(II) acetate, palladium(II) chloride, bisacetonitriledichloropalladium(0), bis(dibenzylideneacetone)palladium(0), tris(dibenzylideneacetone)dipalladium(0) and [1,1′-bis(diphenylphosphino)ferrocene]palladium(II) dichloride

This reaction can be carried out in the presence of a suitable additive agent. Examples of such additive agents include triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl and triphenylarsine.

The reaction can be carried out at a temperature of from 0 to 200° C., more preferably from 20 to 120° C. Reaction times are, in general, from 5 minutes to 48 hours, more preferably from 30 minutes to 24 hours.

Step 2B: In this step, a compound of formula (VII) can be prepared by the alkylation of a compound of formula (VI) under, for example, known alkylating condition such as methylmagnesiumbromide, methylmagnesiumchloride or methyl lithium in an inert solvent. Example of suitable inert organic solvents include: ethers such as diethyl ether, tetrahydrofuran or 1,4-dioxane; dimethylformamide; and halogenated hydrocarbons, such as dichloromethane, dichloroethane or chloroform; or mixtures thereof. The reaction can be carried out at a temperature in the range of from −78 to 100° C., preferably in the range of from 0 to 60° C. Reaction times are, in general, from 10 minutes to 4 days, preferably from 30 minutes to 24 hours.

Step 2C: In this step, a compound of formula (VIII) can be prepared by halogenating a compound of formula (VII) with a halogenating reagent in an inert solvent.

Suitable halogenating reagents include, for example, bromine, chlorine, iodine, N-chlorosuccinimide, N-bromosuccinimide, 1,3-dibromo-5,5-dimethylhydantoin, bis(dimethylacetamide)hydrogen tribromide, tetrabutylammonium tribromide, bromodimethylsulfonium bromide, hydrogen bromide-hydrogen peroxide, nitrodibromoacetonitrile and copper(II) bromide. Examples of suitable inert organic solvents include: ethers such as diethyl ether, tetrahydrofuran and 1,4-dioxane; dimethylformamide; and halogenated hydrocarbons, such as dichloromethane, dichloroethane and chloroform; or mixtures thereof.

The reaction can be carried out at a temperature in the range of from −78 to 100° C., preferably in the range of from 0 to 60° C. Reaction times are, in general, from 10 minutes to 4 days, preferably from 30 minutes to 24 hours.

Step 2D: In this step, a compound of formula (IX) can be prepared by N,N-diformylamination of a compound of formula (VIII) in an inert solvent.

Example of suitable reagents include diformylimido, sodium salt; diformylimido, potassium salt; and diformylimido, lithium salt. Suitable inert organic solvents include: ethers such as diethyl ether, tetrahydrofuran and dioxane; dimethylformamide; and halogenated hydrocarbons, such as dichloromethane, dichloroethane and chloroform; or mixtures thereof. The reaction can be carried out at a temperature in the range of from −78 to 100° C., preferably in the range of from 0 to 60° C. Reaction times are, in general, from 10 minutes to 4 days, preferably from 30 minutes to 24 hours.

Step 2E: In this step, a compound of formula (II) can be prepared by deformylation of a compound of formula (IX) under acidic conditions.

Examples of suitable solvents include co-solvents selected from: water; tetrahydrofuran; 1,4-dioxane; N,N-dimethylformamide; acetonitrile; alcohols, such as methanol and ethanol; halogenated hydrocarbons, such as dichloromethane, 1,2-dichloroethane, chloroform and carbon tetrachloride; and dimethoxyethane.

Example of suitable reagents include acids such as hydrochloric acid, acetic acid and trifluoromethanesulfonic acid. The reaction can be carried out at a temperature in the range of from −78 to 100° C., preferably in the range of from 0 to 60° C. Reaction times are, in general, from 10 minutes to 4 days, preferably from 30 minutes to 24 hours.

Alternatively, according to a third process, when Ar is

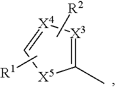

compounds of formula (II) may be prepared from compounds of formula (X) as illustrated by Scheme 3

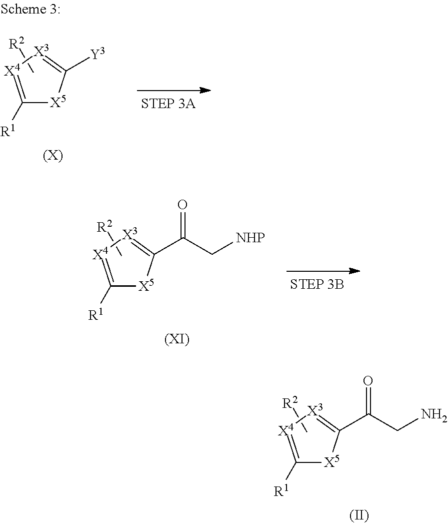

wherein Y³ represents a suitable leaving group such, as a sulfoxy group or a halogen atom, for example chlorine; and P represents a suitable amine protecting group such as those described in Protective Groups in Organic Synthesis edited by T. W. Greene et al. (John Wiley & Sons, 1991).

Step 3A: In this step, a compound of the formula (XI) can be prepared by acylation of a compound of formula (X) under metalation conditions with an alkali metal and acylating reagent in an inert solvent.

Suitable reagents include N-(tert-butoxycarbonyl)glycine N′-methoxy-N′-methylamide. Examples of suitable alkali metal include sodium, potassium, lithium, cesium, rubidium and francium. Examples of suitable solvents include: ethers such as diethylether, tetrahydrofuran and 1,4-dioxane; N,N-dimethylformamide; toluene; acetonitrile; halogenated hydrocarbons, such as dichloromethane, 1,2-dichloroethane, chloroform and carbon tetrachloride; and dimethoxyethane.

The reaction can be carried out at a temperature of from −78 to 200° C., more preferably from 0 to 120° C. Reaction times are, in general, from 5 minutes to 48 hours, more preferably from 30 minutes to 24 hours.

Step 3B: In this Step, the desired compound of formula (II) may be prepared by deprotection of a compound of formula (XI) according to known procedures such as those described in Protective Groups in Organic Synthesis edited by T. W. Greene et al. (John Wiley & Sons, 1991).

Removal of the protecting groups may be carried out under, for example, known hydrogenolysis conditions in the presence of a metal catalyst under hydrogen atmosphere or in the presence of hydrogen sources such as formic acid or ammonium formate in an inert solvent. If desired, the reaction may be carried out under acidic conditions, for example, in the presence of hydrochloric acid or acetic acid. A preferred metal catalyst is selected from, for example, palladium-carbon, palladiumhydroxide-carbon, platinumoxide, platinum-carbon, ruthenium-carbon, rhodium-aluminumoxide, tris[triphenyphosphine] rhodiumchloride. Examples of suitable inert aqueous or non-aqueous organic solvents include: alcohols, such as methanol and ethanol; ethers, such as tetrahydrofuran and 1,4-dioxane; acetone; dimethylformamide; halogenated hydrocarbons, such as dichloromethane, dichloroethane and chloroform; and acetic acid; or mixtures thereof.

The reaction may be carried out at a temperature in the range of from 20 to 100° C., preferably in the range of from 20 to 60° C. Reaction times are, in general, from 10 minutes to 48 hours, preferably from 30 minutes to 24 hours. This reaction may be carried out under a hydrogen atmosphere at a pressure ranging from 1 to 100 atom, preferably from 1 to 10 atom.

According to a fourth process, compounds of formula (III) may be prepared from compounds of formula (XII) as illustrated by Scheme 4.

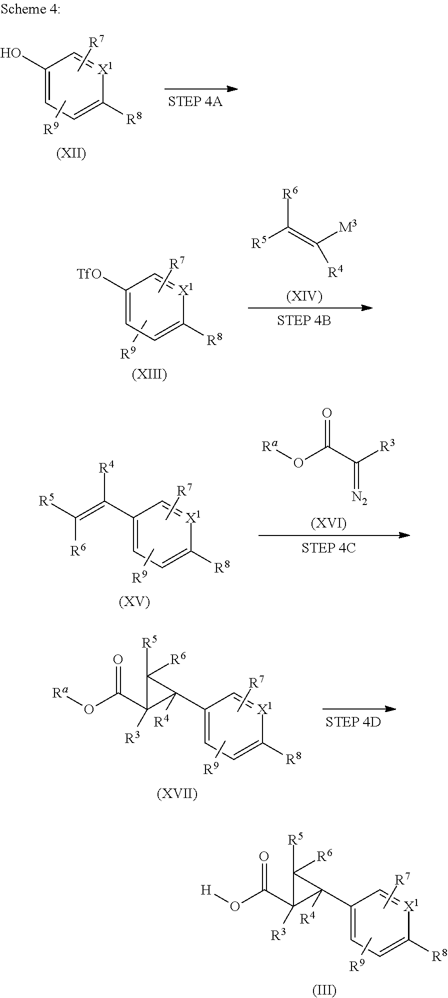

In the above formula, R^{a} represents a suitable protecting group such as (C₁-C₄)alkyl or benzyl; and M³ represents tributylstannane, trimethylstannane, triphenylstannane, tributylsilane, trimethylsilane, triphenylsilane, diphenylborane, dimethylboronate, magnesium bromide and the like.

Step 4A: In this step, a compound of formula (XIII) can be prepared by treating a compound of formula (XII) with trifluoromethane sulfonic acid anhydrate under basic conditions in an inert solvent.

A preferred base is selected from, for example, but not limited to: an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride and potassium hydride; or an amine such as triethylamine, tributylamine, diisopropylethylamine, 2,6-lutidine, pyridine and dimethylaminopyridine.

Examples of suitable solvents include: toluene; xylene; dimethoxyethane; dimethylsulfoxide; tetrahydrofuran; 1,4-dioxane; N,N-dimethylformamide; acetonitrile; halogenated hydrocarbons, such as dichloromethane, 1,2-dichloroethane, chloroform and carbon tetrachloride; and diethylether.

Reaction temperatures are generally in the range of from −78 to 200° C., preferably in the range of from 0° C. to room temperature. Reaction times are, in general, from 1 minute to a day, preferably from 1 hour to 20 hours.

Step 4B: In this step, a compound of formula (XV) can be prepared by treating a compound of a formula (XIII) with a compound of formula (XIV) in the presence of a transition metal catalyst and vinyl metal, vinyl acetate or vinyl methyl ether reagent in an inert solvent.

Examples of suitable solvents include: tetrahydrofuran; 1,4-dioxane; N,N-dimethylformamide; acetonitrile; alcohols, such as methanol and ethanol; halogenated hydrocarbons, such as dichloromethane, 1,2-dichloroethane, chloroform and carbon tetrachloride; and diethylether. The reaction may be carried out in the presence or absence of basic water such as aqueous KOH, NaOH, LiOH or K₂CO₃. Suitable reagents include, for example, metal vinyl reagents such as tributylvinylstannane, trimethylvinylstannane, triphenylvinylstannane, tributylvinylsilane, trimethylvinylsilane, triphenylvinylsilane, diphenylvinylborane, dimethylvinylboronate and vinylmagnesium bromide.

This reaction can be carried out in the presence of a suitable catalyst. There is likewise no particular restriction on the nature of the catalyst used, and any catalyst commonly used in reactions of this type can equally be used here. Examples of such catalysts include those described for step 2A of Scheme 2.

This reaction can be carried out in the presence of a suitable additive agent. Examples of such additive agents include triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, tetrabutylammonium chloride, tetrabutylammonium fluoride, lithium acetate, lithium chloride, triehylamine, potassium sodium methoxide, sodium hydroxide, carbonate, sodium bicarbonate and sodium iodide.

The reaction can be carried out at a temperature of from 0 to 200° C., more preferably from 20 to 120° C. Reaction times are, in general, from 5 minutes to 96 hours, more preferably 30 minutes to 24 hours.

Step 4C: In this step, a compound of formula (XVII) can also be prepared by treating a compound of formula (XV) with a compound of formula (XVI) and a diazo reagent in an inert solvent.

Examples of suitable solvents include: diglyme; dimethylsulfoxide; dimethoxyethane; tetrahydrofuran; 1,4-dioxane; N,N-dimethylformamide; acetonitrile; halogenated hydrocarbons, such as dichloromethane, 1,2-dichloroethane, chloroform and carbon tetrachloride; and acetic acid. Suitable diazo reagents include, for example, diazonium esters such as methyl diazoacetate, ethyl diazoacetate and benzyl-diazoacetate.

This reaction can be carried out in the presence of a suitable catalyst. There is likewise no particular restriction on the nature of the catalyst used, and any catalyst commonly used in reactions of this type can equally be used here. Examples of such catalysts include: Rh(II)acetate, Ru₂(OAc)₄Cl, RuCl₂(PPh₃)(p-cymene), Cu(0), Cu(acetylacetonate)₂, 5,10,15,20-tetraphenyl-21H,23H-porphine Co(II) (Co(TPP)), Pd(OAc)₂.

This reaction can be carried out in the presence of a suitable additive agent. Examples of such additive agents include triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, tetrabutylammonium chloride, tetrabutylammonium fluoride, lithium acetate, lithium chloride, N-methylimidazole, triehylamine, potassium sodium methoxide, sodium hydroxide, carbonate, sodium bicarbonate and sodium iodide.

The reaction can be carried out at a temperature of from 0 to 200° C., more preferably from 20 to 120° C. Reaction times are, in general, from 5 minutes to 96 hours, more preferably from 30 minutes to 24 hours.

Step 4D: In this Step, an acid compound of formula (III) can be prepared by hydrolysis of an ester compound of formula (XVII) in an inert solvent.

The hydrolysis can be carried out by conventional procedures. In a typical procedure, the hydrolysis carried out under basic conditions, e.g. in the presence of sodium hydroxide, potassium hydroxide or lithium hydroxide. Suitable solvents include, for example: alcohols such as methanol, ethanol, propanol, butanol, 2-methoxyethanol, and ethylene gylcol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). Preferred solvents are methanol, ethanol, propanol, tetrahydrofuran (THF), dimethoxyethane (DME), 1,4-dioxane, N,N-dimethylformamide (DMF), and dimethyl sulfoxide (DMSO).

This reaction can be carried out at a temperature in the range of from −20 to 100° C., usually from 20 to 65° C. for from 30 minutes to 24 hours, usually from 60 minutes to 10 hour.

The hydrolysis can alternatively be carried out under acidic conditions, e.g. in the presence of hydrogen halides, such as hydrogen chloride and hydrogen bromide; sulfonic acids, such as p-toluenesulfonic acid and benzenesulfonic acid; pyridium p-toluenesulfonate; or carboxylic acids, such as acetic acid and trifluoroacetic acid. Suitable solvents include, for example: alcohols such as methanol, ethanol, propanol, butanol, 2-methoxyethanol, and ethylene glycol; ethers such as tetrahydrofuran (THF), 1,2-dimethoxyethane (DME), and 1,4-dioxane; amides such as N,N-dimethylformamide (DMF) and hexamethylphospholictriamide; and sulfoxides such as dimethyl sulfoxide (DMSO). Preferred solvents are methanol, ethanol, propanol, tetrahydrofuran (THF), dimethoxyethane (DME), 1,4-dioxane, N,N-dimethylformamide (DMF), and dimethyl sulfoxide (DMSO). This reaction can be carried out at a temperature in the range of from −20 to 100° C., usually from 20 to 65° C. for from 30 minutes to 24 hours, usually from 60 minutes to 10 hour.

According to a fifth process, compounds of formula (XV) may be prepared from compounds of formula (XVII) as illustrated by Scheme 5.

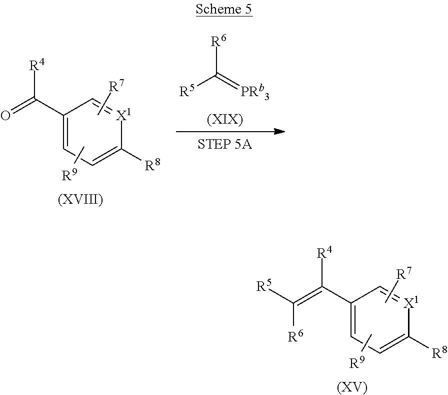

wherein, R^{b} represents (C₁-C₆)alkyl or aryl, such as phenyl.

Step 5A: In this step, a compound of formula (XV) can be prepared by olefination of a compound of formula (XVIII) using phosphinilide (XIX) prepared in situ or phosphorane under standard olefination conditions in an inert solvent or under basic conditions in an inert solvent.

Examples of suitable solvents include: toluene; benzene; xylene; diglyme; dimethylsulfoxide; dimethoxyethane; ethers such as tetrahydrofuran, diethylether and 1,4-dioxane; N,N-dimethylformamide; acetonitrile; alcohols, such as methanol and ethanol; halogenated hydrocarbons, such as dichloromethane, 1,2-dichloroethane, chloroform and carbon tetrachloride; and acetic acid. Suitable phosphine reagents include, for example, triphenylphosphine and tributylphosphine. Suitable methylenehalide reagents include, for example, methyl bromide, ethyl bromide, methyl iodide, ethyl iodide, methyl chloride, ethyl chloride, methyl bromoacetate, bromoacetonitrile, 1-bromoacetone, ethylidene(triphenyl)phosphorane, (triphenylphosphoranylidene)acetonitrile, methyl (triphenylphosphoranylidene)acetate.

A preferred base is selected from, for example, but not limited to, an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride, or an amine such as triethylamine, tributylamine, diisopropylethylamine, 2,6-lutidine, pyridine or dimethylaminopyridine.

The reaction can be carried out at a temperature of from 0 to 300° C., more preferably from 20 to 200° C. Reaction times are, in general, from 5 minutes to 96 hours, more preferably from 30 minutes to 24 hours.

Alternatively, according to a sixth process, compounds of formula (III) may be prepared from compounds of formula (XX) as illustrated by Scheme 6.

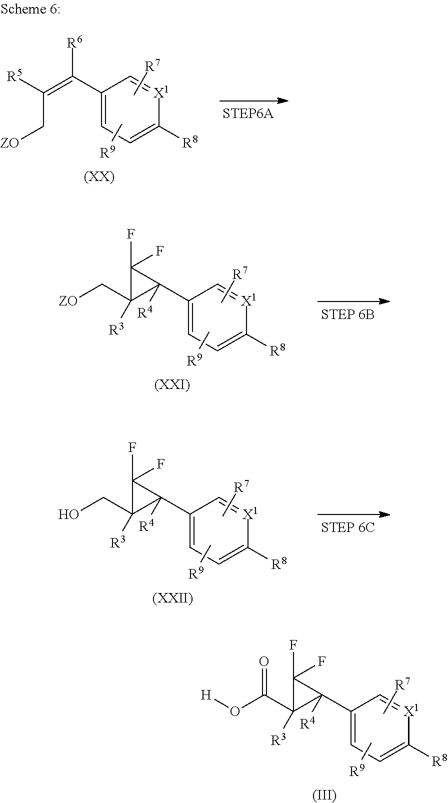

wherein, Z represents a suitable hydroxy protecting group such as (C₁-C₄)alkyl, (C₁-C₁₀)alkylC(═O) or benzyl.

Step 6A: In this step, a compound of formula (XXI) can be prepared by reaction of a compound of formula (XX) with sodium chlorodifluoroacetic acid using a carbene reagent prepared in situ in an inert solvent.

Examples of suitable solvents include: diglyme; dimethylsulfoxide; dimethoxyethane; ethers such as tetrahydrofuran, diethylether and 1,4-dioxane; N,N-dimethylformamide; acetonitrile; alcohols, such as methanol and ethanol; halogenated hydrocarbons, such as dichloromethane, 1,2-dichloroethane, chloroform and carbon tetrachloride; and acetic acid. Suitable reagents include, for example, CH₂I₂, CHCl₃, sodium chlorodifluoroacetate, trimethylsilyl fluorosulfonyldifluoroacetate, trimethylsulfoxonium iodide and diazomethane.

This reaction can be carried out in the presence or absence of a suitable catalyst. There is likewise no particular restriction on the nature of the catalyst used, and any catalyst commonly used in reactions of this type can equally be used here. Examples of such catalysts include: Zn(0), Cu(0), Cu(acetylacetonate)₂, 5,10,15,20-tetraphenyl-21H,23H-porphine Co(II) (Co(TPP)) and Pd(OAc)₂.

This reaction can be carried out in the presence of a suitable additive agent. Examples of such additive agents include, acetylchloride, methylbenzoate, sodium fluoride, triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, sodium hydride, potassium hydride, sodium methoxide and lithium diisopropyl amide.

The reaction can be carried out at a temperature of from 0 to 300° C., more preferably from 20 to 200° C. Reaction times are, in general, from 5 minutes to 96 hours, more preferably from 30 minutes to 24 hours.

Step 6B: In this step, a compound of formula (XXII) can be prepared by deprotection of a compound of formula (XXI) under acidic conditions. Reaction temperatures are generally in the range of 0 to 200° C., preferably room temperature. Reaction times are, in general, from 1 minute to 24 hours, preferably from 5 minutes to 1 hour. Suitable reagents include, for example, hydrochloric acid, trifluoromethane sulfonic acid, methansulfonic acid, p-toluene sulfonic acid and acetic acid.

Examples of suitable solvents include: tetrahydrofuran; 1,4-dioxane; N,N-dimethylformamide; acetonitrile; alcohols, such as methanol and ethanol; halogenated hydrocarbons, such as dichloromethane, 1,2-dichloroethane, chloroform and carbon tetrachloride; and acetic acid.

Alternatively, deprotection may be carried out by a hydrogenation reaction in the presence of a metal catalyst under a hydrogen atmosphere or in the presence of hydrogen sources such as formic acid or ammonium formate in an inert solvent. If desired, the reaction is carried out under acidic conditions, for example, in the presence of hydrochloric acid or acetic acid. A preferred metal catalyst is selected from, for example: nickel catalysts such as Raney nickel; palladium-carbon; palladiumhydroxide-carbon; platinumoxide; platinum-carbon; ruthenium-carbon; rhodium-aluminumoxide; and tris[triphenyphosphine] rhodiumchloride. Examples of suitable inert aqueous or non-aqueous organic solvents include: alcohols, such as methanol and ethanol; ethers, such as tetrahydrofuran and 1,4-dioxane; acetone; dimethylformamide; halogenated hydrocarbons, such as dichloromethane, dichloroethane and chloroform; and acetic acid; or mixtures thereof. The reaction can be carried out at a temperature in the range of from 20 to 100° C., preferably in the range of from 20 to 60° C. Reaction times are, in general, from 10 minutes to 4 days, preferably from 30 minutes to 24 hours. This reaction can be carried out under a hydrogen atmosphere at a pressure ranging from 1 to 100 atom, preferably from 1 to 10 atom.

Step 6C: In this step, a compound of formula (III) can be prepared by oxidation of a compound of a formula (XXII) using an oxidizing agent in an inert solvent.

Examples of suitable oxidizing agents include oxalyl chloride-dimethylsulfoxide (Swern oxidation conditions), pyridinium chlorochromate (PCC), pyridinium dichromate (PDC), manganese dioxide and tetrapropylammonium perruthenate (TPAP). This reaction can be carried out in a suitable inert solvent such as halogenated hydrocarbons such as chloroform, dichloroethane and 1,2-dichloroethane. The reaction may be carried out at a temperature in the range of from −100 to 80° C., usually from −80 to 50° C. for from 5 minutes to 30 hours, usually from 15 minutes to 20 hours.

Alternatively, according to a seventh process, compounds of formula (III) may be prepared from compounds of formula (XXIII) as illustrated by Scheme 7.

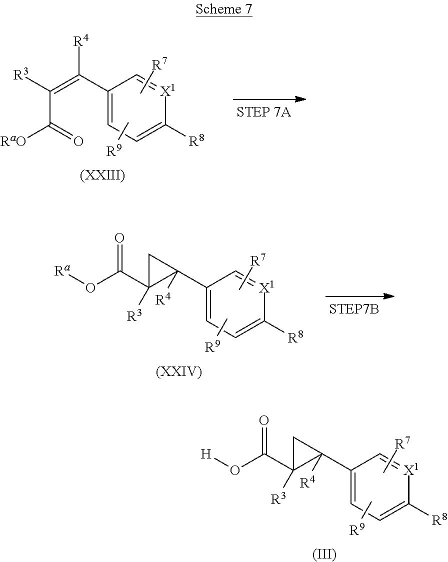

wherein R^{a} represents a suitable acid protecting group such as (C₁-C₄)alkyl or benzyl.

Step 7A: In this step, a compound of formula (XXIV) can be prepared by cyclopropanation of a compound of formula (XXIII) using a carbene prepared in situ in an inert solvent.

Examples of suitable solvents include: diglyme; dimethylsulfoxide; dimethoxyethane; ethers such as tetrahydrofuran, diethylether and 1,4-dioxane; N,N-dimethylformamide; acetonitrile; alcohols, such as methanol and ethanol; halogenated hydrocarbons, such as dichloromethane, 1,2-dichloroethane, chloroform and carbon tetrachloride; and acetic acid. Suitable reagents include, for example, CH₂I₂, CHCl₃, sodium chlorodifluoroacetate, trimethylsilyl fluorosulfonyldifluoroacetate, trimethylsulfoxonium iodide and diazomethane.

This reaction can be carried out in the presence or absence of a suitable catalyst. There is likewise no particular restriction on the nature of the catalyst used, and any catalyst commonly used in reactions of this type can equally be used here. Examples of such catalysts include: Zn(0), Cu(0), Cu(acetylacetonate)₂, 5,10,15,20-tetraphenyl-21H,23H-porphine Co(II) (Co(TPP)) and Pd(OAc)₂.

This reaction can be carried out in the presence of a suitable additive agent. Examples of such additive agents include acetylchloride, methylbenzoate, sodium fluoride, triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, sodium hydride, potassium hydride, sodium methoxide and lithium diisopropyl amide.

The reaction can be carried out at a temperature of from 0 to 300° C., more preferably from 20 to 200° C. Reaction times are, in general, from 5 minutes to 96 hours, more preferably from 30 minutes to 24 hours.

Step 7B: In this step, a compound of formula (III) can be prepared by hydrolysis of an ester compound of formula (XXIV). This reaction analogous to, and may be carried out in the same manner as, and using the same reagents and reaction conditions as described for Step 4D in Scheme 4.

The starting materials in the aforementioned general syntheses are commercially available or may be obtained by conventional methods known to those skilled in the art. Alternatively, certain phenols of formula (XII), when X¹ is CH or CR⁷ and R⁸ is tert-butyl or 2,2,2-trifluoro-1,1-dimethylethyl, may be prepared according to the process illustrated by Scheme 8 below.

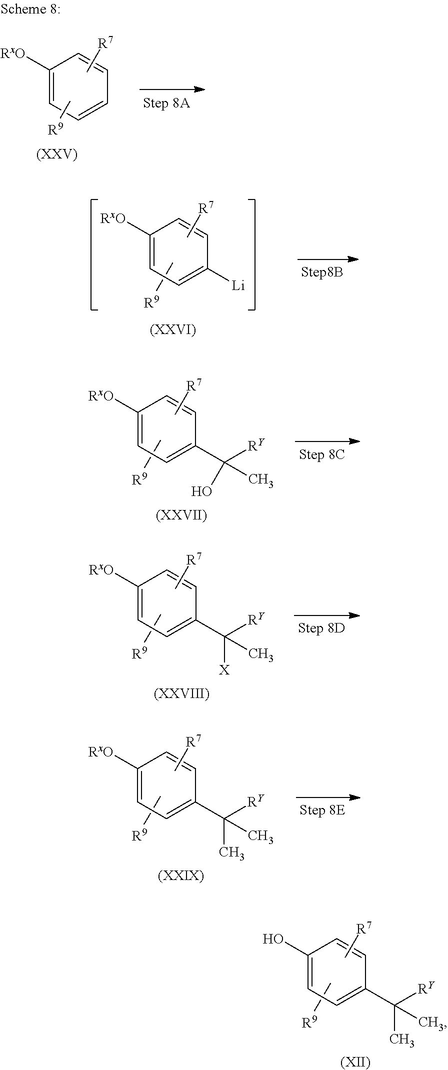

wherein R^{x} is a suitable protecting group such as (C₁-C₆)alkyl, benzyl, benzoyl or (C₁-C₆)alkylsilyl, and is preferably methyl; R^{y} is methyl or trifluoromethyl; and X is halogen.

Step 8A: In this Step, an organolithium compound of formula (XXVI) can be prepared by a directed metalation reaction of a compound of formula (XXV) with an alkyllithium. This reaction may be carried out in the presence of an organometallic reagent or metal. Examples of suitable organometallic reagents include; alkyllithiums such as n-butyllithium, sec-butyllithium and tert-butyllithium; and aryllithiums, such as phenyllithium and lithium naphthalide. Preferred reaction inert solvents include, for example, hydrocarbons, such as hexane; ethers, such as diethyl ether, diisopropyl ether, dimethoxyethane (DME), tetrahydrofuran (THF) and 1,4-dioxane; or mixtures thereof. Reaction temperatures are generally in the range of from −100 to 50° C., preferably in the range of from −100° C. to room temperature. Reaction times are, generally, from 1 minute to a day, preferably from 1 hour to 10 hours.

Step 8B: In this step, a compound of formula (XXVII) can be prepared by the nucleophilic addition of a compound of formula (XXVI) with a ketone. Examples of suitable ketone reagents include acetone and 1,1,1-trifluoroacetone. Preferred inert solvents include, for example, hydrocarbons, such as hexane; ethers, such as diethyl ether, diisopropyl ether, dimethoxyethane (DME), tetrahydrofuran (THF) and dioxane; or mixtures thereof. Reaction temperatures are generally in the range of from −100 to 50° C., preferably in the range of from −100° C. to room temperature. Reaction times are, in general, from 1 minute to a day, preferably from 1 hour to 10 hours.

Step 8C: In this step, a compound of formula (XXVIII) can be prepared by the halogenation reaction of a compound of formula (XXVII) with a halogenating agent. The halogenation may be carried out in the present of a suitable halogenating agent in an inert solvent or without solvent. Preferred inert solvents include, for example, hydrocarbons, such as benzene, toluene, xylene; halogenated hydrocarbons, such as dichloromethane, 1,2-dichloroethane, chloroform or carbon tetrachloride; or mixtures thereof. A preferred halogenating agent is selected from, but is not limited to, the following examples thionyl chloride, oxalyl chloride, phosphorus oxychloride, titanium chloride, phosphorus pentachloride, and is optionally combined with catalytic pyridine. Preferably the halogenating agent is the combination of thionyl chloride and catalytic pyridine. Reaction temperatures are generally in the range of from −100 to 200° C., preferably in the range of from −40 to 100° C. Reaction times are, generally, from 1 minute to a day, preferably from 1 hour to 10 hours.

Step 8D: In this Step, a compound of formula (XXIX) can be prepared by a substitution reaction of a compound of formula (XXVIII) with an alkylating agent. The alkylation may be carried out in the presence of a suitable alkylating agent in an inert solvent. Preferred inert solvents include, for example, halogenated hydrocarbons, such as dichloromethane, 1,2-dichloroethane, chloroform or carbon tetrachloride; ethers, such as diethyl ether, diisopropyl ether, DME, THF)and 1,4-dioxane; hydrocarbons, such as n-hexane, cyclohexane, benzene, toluene; or mixtures thereof. A preferred alkylating agent is selected from, but is not limited to, the following examples trialkylmetal such as trimethylaluminum, triethylaluminum; alkylmagnesium halide, such as methylmagnesium bromide, in the presence of additive compound such as lithium bromide; dialkylzinc halide such as dimethylzinc dichloride prepared from dimethylzinc and titanium chloride; and is preferably trimethylaluminum. Reaction temperatures are generally in the range of from −100 to 200° C., preferably in the range of from −40 to 100° C. Reaction times are, generally, from 1 minute to a day, preferably from 1 hour to 10 hours.

Step 8E: In this Step, a compound of formula (XII) can be prepared by deprotection of a compound of formula (XXIX) with a deprotection agent in an inert solvent. Examples of suitable deprotection agents include: boron halide such as boron tribromide, boron trichloride; and hydrogen halide, such as hydrogen bromide. Preferred inert solvents include, for example, halogenated hydrocarbons such as dichloromethane, 1,2-dichloroethane, chloroform or carbon tetrachloride; and acetic acid. Reaction temperatures are generally in the range of from −100 to 200° C., preferably in the range of from −80 to 80° C. Reaction times are, generally, from 1 minute to a day, preferably from 1 hour to 10 hours.

The compounds of formula (I), and the intermediates mentioned above in the preparation methods can be isolated and purified by conventional procedures, such as recrystallization or chromatographic purification.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product.

### Methods for Assessing Biological Activity

### Human VR1 Antagonist Assay

VR1 antagonistic activity can be determined by the Ca²⁺ imaging assay using human VR1 highly expressing cells. The cells that highly express human VR1 receptors are obtainable from several different conventional methods. The one standard method is cloning from human Dorsal Root Ganglion (DRG) or kidney according to the methods such as described in the journal article; Nature, 389, pp 816-824, 1997. Alternatively VR1 receptors highly expressing human keratinocytes are also known and published in the journal article (Biochemical and Biophysical Research Communications, 291, pp 124-129, 2002). In this article, human keratinocytes demonstrated VR1 mediated intracellular Ca²⁺ increase by addition of capsaicin. Further more, the method to up regulate human VR1 gene, which is usually a silent gene or don't produce detectable level of VR1 receptors, is also available to obtain propriety cells. Such genetic modification method was described in detail; Nat. Biotechnol., 19, pp 440-445, 2001.

The cells that express human VR1 receptors were maintained in culture flask at 37° C. in an environment containing 5% CO₂ until use in the assay. The intracellular Ca²⁺ imaging assay to determine VR1 antagonistic activities were done by following procedures.

The culture medium was removed from the flask and fura-2/AM fluorescent calcium indicator was added to the flask at a concentration of 5 μM in the medium. The flask was placed in CO₂ incubator and incubated for 1 hour. Then the cells expressing the human VR1 receptors were detached from the flask follow by washing with phosphate buffer saline, PBS(−) and re-suspended in assay buffer. The 80 μl of aliquot of cell suspension (3.75×10⁵ cells/ml) was added to the assay plate and the cells were spun down by centrifuge (950 rpm, 20° C., 3 minutes).

### Capsaicin Stimulation Assay:

The capsaicin-induced changes in the intracellular calcium concentration were monitored using FDSS 6000 (Hamamatsu Photonics, Japan), a fluorometric imaging system. The cell suspension in Krebs-Ringer HEPES (KRH) buffer (115 mM NaCl, 5.4 mM KCl, 1 mM MgSO₄, 1.8 mM CaCl₂, 11 mM D-Glucose, 25 mM HEPES, 0.96 mM Na₂HPO₄, pH 7.3) were pre-incubated with varying concentrations of the test compounds or KRH buffer (buffer control) for 15 minutes at room temperature under the dark condition. Then capsaicin solution, which gives 300 nM in assay mixture, was automatically added to the assay plate by the FDSS 6000.

### Acid Stimulation Assay:

The Acid-induced changes in the intracellular calcium concentration were monitored using FDSS 6000 (Hamamatsu Photonics, Japan), a fluorometric imaging system. The cell suspension in resting buffer (HBSS supplemented with 10 mM HEPES, pH 7.4) were pre-incubated with varying concentrations of the test compounds or resting buffer (buffer control) for 15 minutes at room temperature under the dark condition. The cells were automatically added the stimulating solution (HBSS supplemented with MES, final assay buffer pH5.8) by the FDSS 6000. The IC₅₀ values of VR1 antagonists were determined from the half of the increase demonstrated by buffer control samples after acidic stimulation.

### Determination of Antagonist Activity

The monitoring of the changes in the fluorescence signals (λex=340 nm/380 nm, λem=510-520 nm) was initiated at 1 minute prior to the addition of capsaicin solution or acidic buffer and continued for 5 minute. The IC₅₀ values of VR1 antagonists were determined from the half of the increase demonstrated by buffer control samples after agonist stimulation.

### Chronic Contriction Injury Model (CCl Model):

Male Sprague-Dawley rats (270-300 g; B.W., Charles River, Tsukuba, Japan) were used. The chronic constriction injury (CCl) operation was performed according to the method described by Bennett and Xie (Bennett, G. J. and Xie, Y. K. Pain, 33:87-107, 1988). Briefly, animals were anesthetized with sodium pentobarbital (64.8 mg/kg, i.p.) and the left common sciatic nerve was exposed at the level of the middle of the thigh by blunt dissection through biceps femoris. Proximal to the sciatic's trifurcation was freed of adhering tissue and 4 ligatures (4-0 silk) were tided loosely around it with about 1 mm space. Sham operation was performed as same as CCl surgery except for sciatic nerve ligation. Two weeks after surgery, mechanical allodynia was evaluated by application of von Frey hairs (VFHs) to the plantar surface of the hind paw. The lowest amount of force of VFH required to elicit a response was recorded as paw withdrawal threshold (PWT). VFH test was performed at 0.5, 1 and 2 hr post-dosing. Experimental data were analyzed using Kruskal-Wallis test followed by Dunn's test for multiple comparisons or Mann-Whitney U-test for paired comparison.

### Half-Life in Human Liver Microsomes (HLM)

Test compounds (1 μM) were incubated with 3.3 mM MgCl₂ and 0.78 mg/mL HLM (HL101) in 100 mM potassium phosphate buffer (pH 7.4) at 37° C. on the 96-deep well plate. The reaction mixture was split into two groups, a non-P450 and a P450 group. NADPH was only added to the reaction mixture of the P450 group. An aliquot of samples of P450 group was collected at 0, 10, 30, and 60 min time point, where 0 min time point indicated the time when NADPH was added into the reaction mixture of P450 group. An aliquot of samples of non-P450 group was collected at −10 and 65 min time point. Collected aliquots were extracted with acetonitrile solution containing an internal standard. The precipitated protein was spun down in centrifuge (2000 rpm, 15 min). The compound concentration in supernatant was measured by LC/MS/MS system.

The half-life value was obtained by plotting the natural logarithm of the peak area ratio of compounds/internal standard versus time. The slope of the line of best fit through the points yields the rate of metabolism (k). This was converted to a half-life value using following equations:

Half-life=ln 2/k

### Mono-Iodoacetate (MIA)-Induced OA Model

Male 6-weeks-old Sprague-Dawley (SD, Japan SLC or Charles River Japan) rats were anesthetized with pentobarbital. Injection site (knee) of MIA was shaved and cleaned with 70% ethanol. Twenty-five μl of MIA solution or saline was injected in the right knee joint using a 29 G needle. The effect of joint damage on the weight distribution through the right (damaged) and left (untreated) knee was assessed using an incapacitance tester (Linton Instrumentation, Norfolk, UK). The force exerted by each hind limb was measured in grams. The weight-bearing (WB) deficit was determined by a difference of weight loaded on each paw. Rats were trained to measure the WB once a week until 20 days post MIA-injection. Analgesic effects of compounds were measured at 21 days after the MIA injection. Before the compound administration, the “pre value” of WB deficit was measured. After the administration of compounds, attenuation of WB deficits was determined as analgesic effects.

### Drug Substance

Pharmaceutically acceptable salts of the compounds of formula (I) include the acid addition and base salts thereof.

Suitable acid addition salts are formed from acids which form non-toxic salts. Examples include acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionization in the salt may vary from completely ionized to almost non-ionized.

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

Hereinafter all references to compounds of formula (I) include references to salts, solvates and complexes thereof and to solvates and complexes of salts thereof.

The compounds of the invention include compounds of formula (I) as hereinbefore defined, polymorphs, prodrugs, and isomers thereof (including optical, geometric and tautomeric isomers) as hereinafter defined and isotopically-labeled compounds of formula (I).

As stated, the invention includes all polymorphs of the compounds of formula (I) as hereinbefore defined.

Also within the scope of the invention are so-called ‘prodrugs’ of the compounds of formula (I). Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985).

Some examples of prodrugs in accordance with the invention include:

(i) where the compound of formula (I) contains an alcohol functionality (—OH), an ether thereof, for example, replacement of the hydrogen with (C₁-C₆)alkanoyloxymethyl; and

(ii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types may be found in the aforementioned references.

Finally, certain compounds of formula (I) may themselves act as prodrugs of other compounds of formula (I).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁸Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colorants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified controlled release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably. to a pH of from 3 to 9), but, for some applications, they may be more suitably formulated as a sterile non-aqueous solution or as powdered a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for use with needle-free injection administration comprise a compound of the invention in powdered form in conjunction with a suitable vehicle such as sterile, pyrogen-free water.

Formulations for parenteral administration may be formulated to be immediate and/or modified controlled release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

### Topical Administration

The compounds of the invention may also be administered topically to the skin or mucosa, that is, dermally or transdermally. Typical formulations for this purpose to include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified controlled release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified controlled release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 μg to 10 mg of the compound of formula (I). The overall daily dose will typically be in the range 1 μg to 10 mg which may be administered in a single dose or, more usually, as divided doses throughout the day.

### Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified controlled release. Modified release formulations include delayed-, sustained-, pulsed-, controlled-, targeted and programmed release.

### Other Technologies

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Dosage

For administration to human patients, the total daily dose of the compounds of the invention is typically in the range 0.1 mg to 3000 mg, preferably from 1 mg to 500 mg, depending, of course, on the mode of administration. For example, oral administration may require a total daily dose of from 0.1 mg to 3000 mg, preferably from 1 mg to 500 mg, while an intravenous dose may only require from 0.1 mg to 1000 mg, preferably from 0.1 mg to 300 mg. The total daily dose may be administered in single or divided doses.

These dosages are based on an average human subject having a weight of about 65 kg to 70 kg. The physician will readily be able to determine doses for subjects whose weight falls outside this range, such as infants and the elderly.

For the avoidance of doubt, references herein to “treatment” include references to curative, palliative and prophylactic treatment.

A VR1 antagonist may be usefully combined with another pharmacologically active compound, or with two or more other pharmacologically active compounds, particularly in the treatment of pain. For example, a VR1 antagonist, particularly a compound of formula (I), or a pharmaceutically acceptable salt or solvate thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or vaiproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g. oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-[(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S42-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl]phenyl}ethyl]amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine;
a 5-HT3 antagonist, such as ondansetron;
   and the pharmaceutically acceptable salts and solvates thereof.

Thus, the invention further provides a combination comprising a compound of the invention or a pharmaceutically acceptable salt or solvate, and a compound or class of compounds selected from the groups listed above. There is also provided a pharmaceutical composition comprising such a combination, together with a pharmaceutically acceptable excipient, diluent or carrier, particularly for the treatment of a disease for which a VR1 antagonist is implicated.

In as much as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### EXAMPLES

The invention is illustrated by the following non-limiting examples in which, unless stated otherwise: all operations were carried out at room or ambient temperature, that is, in the range of from 18 to 25° C.; evaporation of solvent was carried out using a rotary evaporator under reduced pressure with a bath temperature of up to 60° C.; reactions were monitored by thin layer chromatography (TLC) and reaction times are given for illustration only; the structure and purity of all isolated compounds were assured by at least one of the following techniques: TLC (Merck silica gel 60 F₂₅₄ precoated TLC plates), mass spectrometry, nuclear magnetic resonance spectra (NMR), or infrared absorption spectra (IR). Yields are given for illustrative purposes only. Flash column chromatography was carried out using Merck silica gel 60 (230-400 mesh ASTM) or Biotage amino bounded silica (35-75 μm, KP-NH) or Biotage silica (32-63 μm, KP-SiI). Low-resolution mass spectral data (EI) were obtained on a Integrity (Waters) mass spectrometer. Low-resolution mass spectral data (ESI) were obtained on a ZMD (Micromass) mass spectrometer. NMR data was determined at 270 MHz (JEOL JNM-LA 270 spectrometer) or 300 MHz (JEOL JNM-LA300 spectrometer) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, quint=quintet, m=multiplet, br.=broad, etc. IR spectra were measured by a Shimazu infrared spectrometer (IR-470). Chemical symbols have their usual meanings; by (boiling point), mp (melting point), L (liter(s)), mL (milliliter(s)), g (gram(s)), mg (milligram(s)), mol (moles), mmol (millimoles), eq. (equivalent(s)), quant. (quantitative yield), sat. (saturated), aq (aqua).

### Example 1

### 2-(4-Tert-Butylphenyl)-N-[2-(3-Methylpyridin-2-yl)-2-Oxoethyl]Cyclopropanecarboxamide

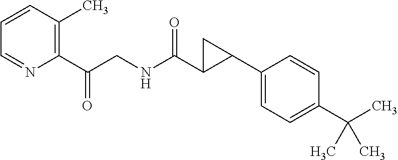

### 1A) 2-(N,N-Diformylamino)-1-(3-Methyl-2-Pyridinyl)Ethanone

To a suspension of 2-bromo-1-(3-methyl-2-pyridinyl)ethanone hydrobromide (495 mg, 1.68 mmol) in acetonitrile (5 ml) was added sodium diformylamide (478 mg, 5.03 mmol) stepwise at room temperature and the mixture was stirred at room temperature for 24 hours. The reaction was partitioned with saturated NaHCO₃ aqueous solution and ethyl acetate, and the organic layer was separated and dried over Na₂SO₄. Then filtration and evaporation under reduced pressure gave the crude residue which was purified by silica gel column chromatography, eluting with ethyl acetate/hexane (1:2), to furnish 2-(N,N-diformylamino)-1-(3-methyl-2-pyridinyl)ethanone (143 mg, 41%) as a white solid.

¹H-NMR (CDCl₃) δ 2.60 (3H, s), 5.36 (2H, s), 7.41 (1H, dd, J=4.6, 7.7 Hz), 7.61-7.64 (1H, m), 8.54-8.56 (1H, m), 9.05 (2H, s).

MS (ESI) m/z 207 (M+H)⁺.

### 1B) 2-Amino-1-(3-Methyl-2-Pyridinyl)Ethanone Dihydrochloride

A solution of 2-(N,N-diformylamino)-1-(3-methyl-2-pyridinyl)ethanone (Example 1A, 142 mg, 0.689 mmol) in ethanol (2 ml) and concentrated HCl (0.5 ml) was stirred at 50° C. for 1 hour. The mixture was concentrated and co-evaporated with toluene. The resulting solid was filtrated and washed with ethyl acetate and diethylether to furnish 2-amino-1-(3-methyl-2-pyridinyl)ethanone dihydrochloride (167 mg, quant.) as a white solid.

¹H-NMR (DMSO-d₆) δ 2.59 (3H, s), 4.20-4.80 (2H, m), 7.65 (1H, dd, J=4.6, 7.8 Hz), 7.90 (1H, d, J=7.8 Hz), 8.46 (2H, NH), 8.62 (1H, d, J=4.3 Hz).

MS (ESI) m/z 151 (M+H)⁺.

### 1C) 2-(4-Tert-Butylphenyl)-N-[2-(3-Methylpyridin-2-yl)-2-Oxoethyl]-Cyclopropanecarboxamide

To the CH₂Cl₂ (10 ml) solution of 2-(4-tert-butylphenyl)cyclopropanecarboxylic acid (61 mg, 0.275 mmol), oxalyl dichloride (72 μl, 0.83 mmol) and N,N-dimethylformamide (DMF) (one drop) were added and the mixture was stirred for 1 hour at room temperature. After evaporation, the crude residue was dried under reduced pressure. Then, a CH₂CH₂ (1 ml) solution of the crude residue described above was added to a CH₂Cl₂ (1 ml) solution of 2-amino-1-(3-methyl-2-pyridinyl)ethanone dihydrochloride (Example 1B, 61 mg, 0.275 mmol) and triethylamine (115 μA 0.83 mmol) and the mixture was stirred for 2 hours at room temperature. The reaction was quenched with saturated NaHCO₃ aqueous solution and the product was extracted with ethyl acetate, and the organic layer was dried over Na₂SO₄. Then, filtration, evaporation and purification by silica gel column chromatography, eluting with hexane/ethyl acetate (1:1), gave 2-(4-tert-butylphenyl)-N-[2-(3-methylpyridine-2-yl)-2-oxoethyl]cyclopropanecarboxamide (20A mg, 22%) as a white solid.

¹H-NMR (300 HMz, CDCl₃) δ 1.31 (9H, s), 1.61-1.80 (3H, m), 2.50-2.55 (1H, m), 2.62 (3H, s), 4.99-5.01 (2H, dd, J=2.6, 4.6 Hz), 6.58 (1H, br), 7.07 (2H, d, J=8.1 Hz), 7.33 (2H, d, J=8.1 Hz), 7.38 (1H, dd, J=4.6, 7.9 Hz), 7.62 (2H, d, J=7.9 Hz), 8.53 (1H, d, J=4.6 Hz).

MS (ESI) m/z 351 (M+H)⁺.

### Example 2

### 2-(4-Tert-Butylphenyl)-N-[2-(5-Methoxy-2-Methylphenyl)-2-Oxoethyl]Cyclopropanecarboxamide

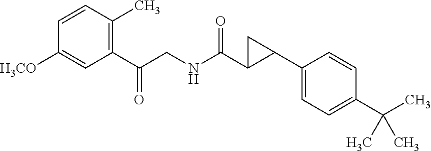

### 2A) 2-(N,N-Diformylamino)-1-(5-Methoxy-2-Methylphenyl)Ethanone

To a solution of 1-(5-methoxy-2-methylphenyl)ethanone (0.328 g, 2.0 mmol), 25% hydrobromic acid in acetic acid (2 ml) and acetic acid (4 ml), bromine (352 mg, 2.2 mmol) was added dropwise at room temperature, and the mixture was stirred at room temperature for 10 hours. After being quenched with saturated NaHCO₃ aqueous solution, the product was extracted with hexane/ethyl acetate (1:1) and dried over Na₂SO₄. Then, filtration and evaporation gave 2-bromo-1-(5-methoxy-2-methylphenyl)ethanone (0.562 mg, quant.), which was used in the next reaction without further purification. To a CH₃CN (2.0 ml) solution of 2-bromo-1-(5-methoxy-2-methylphenyl)ethanone (0.562 mg, crude) was added sodium diformylamide (0.228 g, 2.4 mmol) stepwise at room temperature and the mixture was stirred at room temperature for 24 hours. The reaction was partitioned with saturated NaHCO₃ aqueous solution and ethyl acetate, and the organic layer was separated and dried over Na₂SO₄. Then, filtration and evaporation under reduced pressure gave the crude residue which was purified by silica gel column chromatography, eluting with ethyl acetate/hexane (1:2), to furnish 2-(N,N-diformylamino)-1-(5-methoxy-2-methylphenyl)ethanone (0.224 g, 47% in 2 steps).

MS (ESI) m/z 236 (M+H)⁺.

### 2B) 2-Amino-1-(5-Methoxy-2-Methylphenyl)Ethanone

To an ethanol (4.0 ml) solution of 2-(N,N-diformylamino)-1-(3-methoxy-6-toluoyl)ethanone (Example 2A, 0.224 g, 0.952 mmol), concentrated HCl was added and the mixture was stirred for 1 hour at 50° C. After evaporation of the solvent, crude 2-amino-1-(5-methoxy-2-methylphenyl)ethanone residue was used in the next reaction without further purification.

### 2C) 2-(4-Tert-Butylphenyl)-N-[2-(5-Methoxy-2-Methylphenyl)-2-Oxoethyl]Cyclopropanecarboxamide

To a CH₂Cl₂ (10 ml) solution of 2-(4-tert-butylphenyl)cyclopropanecarboxylic acid (137 mg, 0.627 mmol), oxalyl dichloride (164 μl, 1.88 mmol) and N,N-dimethylformamide (DMF) were added and the mixture was stirred for 1 hour at room temperature. After evaporation of the solvent, the crude residue was dried under reduced pressure. Then, a CH₂CH₂ (1 ml) solution of the crude residue described above was added to a CH₂Cl₂ (1 ml) solution of 2-amino-1-(5-methoxy-2-methylphenyl)ethanone (Example 2B, 135 mg, 0.627 mmol) and diisopropylethylamine (437 μl, 2.51 mmol) and the mixture was stirred for 2 hours at room temperature. The reaction was quenched with saturated NaHCO₃ aqueous solution and the product was extracted with ethyl acetate, and the organic layer was separated and dried over Na₂SO₄. Then, filtration, evaporation and purification by silica gel column chromatography, eluting with hexane/ethyl acetate (1:1), gave 2-(4-tert-butylphenyl)-N-[2-(5-methoxy-2-methylphenyl)-2-oxoethyl]cyclopropanecarboxamide (130 mg, 55%) as a white solid.

¹H NMR (CDCl₃) δ 1.31 (9H, s), 1.60-1.81 (3H, m), 2.46 (3H, s), 2.46-2.54 (1H, m), 3.83 (3H, s), 4.67-4.70 (2H, m), 6.73 (1H, br), 6.97-7.02 (1H, m), 7.07 (2H, d, J=8.1 Hz), 7.19-7.23 (2H, m), 7.33 (2H, d, J=8.1 Hz).

MS (ESI) m/z 380 (M+H)⁺.

### Example 3

### 2-(4-Tert-Butylphenyl)-N-[2-(5-Hydroxy-2-Methylphenyl)-2-Oxoethyl]Cyclopropanecarboxamide

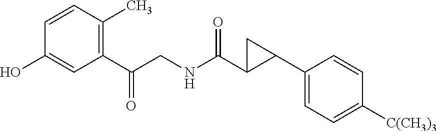

To a CH₂Cl₂ solution of 2-(4-tert-butylphenyl)-N-[2-(5-methoxy-2-methylphenyl)-2-oxoethyl]cyclopropanecarboxamide (Example 2, 65 mg, 0.17 mmol), a 1M CH₂Cl₂ solution of BBr₃ (1 ml) was added and the mixture was stirred for 1 hour at 0° C. The reaction was quenched with saturated NaHCO₃ aqueous solution and the product was extracted with ethyl acetate, the organic layer was separated and dried over Na₂SO₄. Then, filtration, evaporation and purification by silica gel column chromatography, eluting with hexane/ethyl acetate (1:1), gave 2-(4-tert-butylphenyl)-N-[2-(5-hydroxy-2-methylphenyl)-2-oxoethyl]cyclopropanecarboxamide (6.4 mg, 10%) as a white solid.

¹H NMR (CDCl₃) δ 1.31 (9H, s), 1.67-1.86 (3H, m), 2.46 (3H, s), 2.54-2.59 (1H, m), 4.75 (2H, d, J=4.0 Hz), 6.98-7.05 (1H, m), 7.06 (2H, d, J=8.6 Hz), 7.14 (1H, d, J=8.6 Hz), 7.32 (2H, d, J=7.9 hz), 7.42 (1H, d, J=2.6 Hz) 8.56 (1H, brs).

MS (ESI) m/z 366 (M+H)⁺.

### Example 4

### 2-(4-Tert-Butyl-3-Fluorophenyl)-N-[2-(3-Methylpyridin-2-yl)-2-Oxoethyl]Cyclopropanecarboxamide

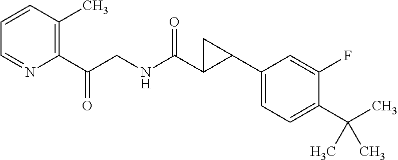

### 4A) 4-Tert-Butyl-3-Fluorophenol

ZrCl₄ (11.7 g, 50 mmol) in CH₂Cl₂ (130 ml), tBuOMe (4.44 g, 50 mmol) and 3-fluorophenol (5.6 g, 50 mmol) were mixed at room temperature and the reaction mixture was stirred for 2 hours at 50° C. The reaction was quenched with H₂O, and the product was extracted with ethyl acetate and dried over MgSO₄. After filtration, evaporation of the solvent gave the crude residue, which was purified by silica gel column chromatography, eluting with gradually from hexane only to hexane/ethyl acetate (9:1), to give 4-tert-butyl-3-fluorophenol (4.25 g, 51%) as a white solid.

¹H NMR (CDCl₃) δ 1.34 (9H, s), 4.97 (1H, brs), 6.56-6.50 (2H, m), 7.13 (1H, t, J=8.7 Hz).

### 4B) 4-Tert-Butyl-3-Fluorophenyl Trifluoromethanesulfonate

To a pyridine (30 ml) and CH₂Cl₂ (50 ml) solution of 4-tert-butyl-3-fluorophenol (Example 4A, 4.25 g, 25 mmol), trifluoromethane sulfonic acid anhydride (10.6 g, 37.5 mmol) and N,N-dimethylaminopyridine (DMAP) (30 mg, 0.25 mmol) were added and the mixture was stirred for 2 hours at 0° C. After being quenched with H₂O, the product was extracted with hexane, evaporated, purified by silica gel column chromatography, eluting with gradually from hexane only to hexane/ethyl acetate (9:1), to give 4-tert-butyl-3-fluorophenyl trifluoromethanesulfonate (6.7 g, 88%) as a colorless oil.

¹H NMR (CDCl₃) δ 1.38 (9H, s), 6.95-7.03 (2H, m), 7.37 (1H, t, J=8.1 Hz)

MS (ESI) m/z 301 (M+H)⁺.

### 4C) 1-Tert-Butyl-2-Fluoro-4-Vinylbenzene

To a N,N-dimethylformamide (DMF) (100 ml) solution of 4-tert-butyl-3-fluorophenyl trifluoromethanesulfonate (Example 4B, 3.27 g, 10.9 mmol), vinyltributylstannane (3.8 g, 12.0 mmol), LiCl (4.62 g, 108 mmol) and Pd(PPh₃)₂Cl₂ (0.383 g, 0.54 mmol) were added and the mixture was stirred for 30 minutes at room temperature, followed by additional stirring for 20 hours at 30° C. The reaction was quenched with H₂O and the product was extracted with hexane. Then, evaporation and purification by silica gel column chromatography, eluting with hexane, gave 1-tert-butyl-2-fluoro-4-vinylbenzene (1.87 g, 96%) as a colorless oil.

### 4D) Ethyl 2-(4-Tert-Butyl-3-Fluorophenyl)Cyclopropanecarboxylate

To a toluene (12 ml) solution of 1-tert-butyl-2-fluoro-4-vinylbenzene (Example 4C, 1.86 g, 10.4 mmol), 5,10,15,20-tetraphenyl-21H,23H-porphine Co(II) (Co(TPP)) (0.21 g, 0.3 mmol) and 1-methyl-1H-imidazole (2.56 g, 31 mmol), ethyldiazoacetate (1.66 g, 14.5 mmol) was added and the mixture was stirred for 5 minutes at room temperature followed by additional stirring for 1 hour at 80° C. Then, evaporation and purification by silica gel column chromatography, eluting with gradually from hexane to hexane/ethyl acetate (10:1), gave ethyl 2-(4-tert-butyl-3-fluorophenyl)cyclopropanecarboxylate (2.13 g, 77%) as a colorless oil.

¹H NMR (CDCl₃) δ 0.88 (3H, t, J=8.1 Hz), 1.24-1.30 (1H, m), 1.35 (9H, s), 1.55-1.62 (1H, m), 1.84-1.90 (1H, m), 2.43-2.50 (1H, m), 4.17 (2H, q, J=8.1 Hz), 6.73 (1H, br, j=8.1 Hz), 6.82 (1H, d, J=8.1 Hz), 7.19 (1H, t, J=8.1 Hz).

MS (ESI) m/z 265 (M+H)⁺.

### 4E) 2-(4-Tert-Butyl-3-Fluorophenyl)Cyclopropanecarboxylic Acid

To a tetrahydrofuran (THF) (5 ml) solution of ethyl 2-(4-tert-butyl-3-fluorophenyl)cyclopropanecarboxylate (Example 4D, 2.13 g, 6.8 mmol), 2N NaOH (10 ml) and methanol (10 ml) were added and the mixture was stirred for 30 minutes at 80° C. After the reaction was completed, the basic mixture was acidified with 2N HCl and the product was extracted with ethyl acetate followed by evaporation of the solvent to give 2-(4-tert-butyl-3-fluorophenyl)cyclopropanecarboxylic acid (1.63 g, 89%) as a white solid.

MS (ESI) m/z 235 (M−H)⁻.

### 4F) 2-(4-Tert-Butyl-3-Fluorophenyl)-N-[2-(3-Methylpyridin-2-yl)-2-Oxoethyl]Cyclopropanecarboxamide

To a tetrahydrofuran (THF) (3.0 ml) solution of (4-tert-butyl-3-fluoro-phenyl)cyclopropane carboxylic acid (Example 4E, 219 mg, 0.93 mmol) was added 2-chloro-1,3-dimethylimidazolinium chloride (CDI) (150 mg, 0.93 mmol) at room temperature and the mixture was stirred for 1 hour at room temperature and then, to this reaction was added Et₃N (2.5 ml) and 2-amino-1-(3-methyl-2-pyridinyl)ethanone dihydrochloride (207 mg, 1.11 mmol). After the mixture was stirred for 10 hours, the reaction was quenched with saturated NaHCO₃ aqueous solution. Then, the product was extracted with ethyl acetate and dried over Na₂SO₄. After filtration, evaporation of the solvent and purification by silica gel column chromatography, eluting with hexane/ethyl acetate/methylene chloride (1:2:2), gave 2-(4-tert-butyl-3-fluorophenyl)-N-[2-(3-methylpyridine-2-yl)-2-oxoethyl]cyclopropanecarboxamide (105 mg, 26%) as a white solid.

¹H-NMR (CDCl₃) δ1.21-1.28 (m, 1H), 1.35 (9H, s), 1.59-1.79 (2H, m), 2.46-2.53, 2.62 (3H, s), 6.61 (1H, br), 6.74 (1H, d, J=10.8 Hz), 6.84 (1H, dd, J=2.7, 8.1 Hz), 7.13-7.22 (1H, m), 7.38 (1H, d, J=5.4 Hz), 7.62 (1H, d, J=8.1 Hz), 8.53 (1H, d, J=5.4 Hz).

MS (ESI) m/z 369 (M+H)⁺.

### Example 5

### N-[2-(3-Methylpyridin-2-yl)-2-Oxoethyl]-2-[4-(2,2,2-Trifluoro-1,1-Dimethylethyl)Phenyl]Cyclopropanecarboxamide

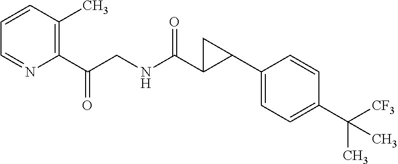

### 5A) 4-(2,2,2-Trifluoro-1,1-Dimethylethyl)Phenyl Trifluoromethanesulfonate

To a pyridine (8 ml) and CH₂Cl₂ (12 ml) solution of 4-(2,2,2-trifluoro-1,1-dimethylethyl)phenol (1.2 g, 6 mmol), trifluoromethane sulfonic acid anhydride (2.54 g, 9 mmol) and N,N-dimethylaminopyridine (DMAP) (12 mg, 0.1 mmol) were added and the mixture was stirred for 3 hours at 0° C. After being quenched with H₂O, the product was extracted with hexane, the solvent was evaporated, and the crude product was purified by silica gel column chromatography, eluting with gradually from hexane only to hexane/ethyl acetate (9:1), to give 4-(2,2,2-trifluoro-1,1-dimethylethyl)phenyl trifluoromethanesulfonate (1.8 g, 89%) as a colorless oil.

¹H NMR (CDCl₃) δ 1.59 (6H, s), 7.28 (2H, d, J=8.1 Hz), 7.59 (2H, d, J=8.1 Hz)

### 5B) 1-(2,2,2-Trifluoro-1,1-Dimethylethyl)-4-Vinylbenzene

To a N,N-dimethylformamide (DMF) (50 ml) solution of 4-(2,2,2-trifluoro-1,1-dimethylethyl)phenyl trifluoromethanesulfonate (Example 5A, 1.80 g, 5.3 mmol), vinyltributylstannane (1.86 g, 5.8 mmol), LiCl (2.25 g, 53 mmol) and Pd(PPh₃)₂Cl₂ (186 mg, 0.26 mmol) were added and the mixture was stirred for 30 minutes at room temperature followed by additional stirring for 10 hours at 28° C. The reaction was quenched with H₂O and the product was extracted with hexane. Then, evaporation and purification by silica gel column chromatography, eluting with hexane, gave 1-(2,2,2-trifluoro-1,1-dimethylethyl)-4-vinylbenzene (0.815 g, 72%) as a colorless oil.

¹H NMR (CDCl₃) δ 1.57 (6H, s), 5.27 (1H, d, J=10.8 Hz), 5.76 (1H, d, J=16.2 Hz), 6.71 (1H, dd, J=10.8, 16.2 Hz), 7.38-7.47 (4H, m).

### 5C) Ethyl 2-[4-(2,2,2-Trifluoro-1,1-Dimethylethyl)Phenyl]Cyclopropanecarboxylate

To a toluene (4 ml) solution of 1-(2,2,2-trifluoro-1,1-dimethylethyl)-4-vinylbenzene (Example 5B, 0.8 g, 3.73 mmol), 5,10,15,20-tetraphenyl-21H,23H-porphine Co(II) (Co(TPP)) (0.075 g, 0.1 mmol) and 1-methyl-1H-imidazole (0.92 g, 11 mmol), ethyldiazoacetate (0.6 g, 5.26 mmol) was added and the mixture was stirred for 5 minutes at room temperature followed by additional stirring for 1 hour at 80° C. Then, evaporation and purification by silica gel column chromatography, eluting with gradually from hexane to hexane/ethyl acetate (10:1), gave ethyl 2-[4-(2,2,2-trifluoro-1,1-dimethylethyl)phenyl]cyclopropanecarboxylate (1.0 g, 89%) as a colorless oil.

¹H NMR (CDCl₃) δ 1.28 (3H, t, J=8.1 Hz), 1.25-1.35 (1H, m), 1.55 (6H, s), 1.55-1.64 (1H, m), 1.87-1.94 (1H, m), 2.47-2.54 (1H, m), 4.17 (2H, q, J=8.1 Hz), 7.10 (2H, d, j=8.1 Hz), 7.41 (2H, d, J=8.1 Hz).

MS (ESI) m/z 301 (M+H)⁺.

### 5D) 2-[4-(2,2,2-Trifluoro-1,1-Dimethylethyl)Phenyl]Cyclopropanecarboxylic Acid

To a tetrahydrofuran (THF) (5 ml) solution of ethyl 2-[4-(2,2,2-trifluoro-1,1-dimethylethyl)phenyl]cyclopropanecarboxylate (Example 5C, 1.0 g, 3.3 mmol), 2N NaOH (3 ml) and methanol (3 ml) were added and the mixture was stirred for 30 minutes at 50° C. After the reaction was completed, the basic mixture was acidified with 2N HCl and the product was extracted with ethyl acetate followed by evaporation of the solvent to give 2-[4-(2,2,2-trifluoro-1,1-dimethylethyl)phenyl]cyclopropanecarboxylic acid (0.82 g, 90%) as a white solid.

MS (ESI) m/z 271 (M−H)⁻.

### 5E) N-[2-(3-Methylpyridin-2-yl)-2-Oxoethyl]-2-[4-(2,2,2-Trifluoro-1,1-Dimethylethyl)Phenyl]Cyclopropanecarboxamide

The procedure as described in Example 4F was performed using 2-[4-(2,2,2-trifluoro-1,1-dimethylethyl)phenyl]cyclopropanecarboxylic acid (Example 5D, 252 mg, 0.93 mmol) as starting material to give N-[2-(3-methylpyridin-2-yl)-2-oxoethyl]-2-[4-(2,2,2-trifluoro-1,1-dimethylethyl)phenyl]cyclopropanecarboxamide (85 mg, 20%) as a white solid.

¹H-NMR (CDCl₃) δ 1.26-1.33 (1H, m), 1.56 (6H, s), 1.64-1.82 (2H, m), 2.50-2.53, 2.57 (1H, m), 2.62 (3H, s), 5.01 (2H, br), 6.59 (1H, br), 7.14 (2H, d, J=8.1 Hz), 7.36-7.43 (3H, m), 7.62 (1H, d, J=8.1 Hz), 8.53 (1H, d, J=5.4 Hz).

MS (ESI) m/z 405 (M+H)⁺.

### Example 6

### 2-(4-Tert-Butylphenyl)-2-Methyl-N-[2-(3-Methylpyridin-2-yl)-2-Oxoethyl]Cyclopropanecarboxamide

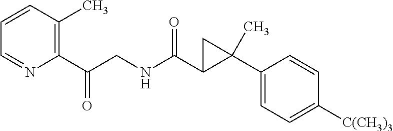

The procedure as described in Example 4F was performed using (4-tert-butylphenyl)-2-methylcyclopropane carboxylic acid (215 mg, 0.93 mmol) as starting material to give 2-(4-tert-butylphenyl)-2-methyl-N-[2-(3-methylpyridine-2-yl)-2-oxoethyl]cyclopropanecarboxamide (30 mg, 10%) as a white solid.

¹H-NMR (CDCl₃) δ 1.33 (9H, s), 1.38-1.44 (m, 1H), 1.52 (3H, s), 5.02 (2H, d, J=2.7 Hz), 6.58 (1H, s), 7.24 (2H, d, J=8.1 Hz), 7.36 (2H, d, J=8.1 Hz), 7.36-7.41 (1H, m), 7.62 (1H, d, J=5.4 Hz), 8.53 (1H, d, J=2.7 Hz).

MS (ESI) m/z 365 (M+H)⁺.

### Example 7

### 2-(4-Tert-Butyl-3-Chlorophenyl)-N-[2-(3-Methylpyridin-2-yl)-2-Oxoethyl]Cyclopropanecarboxamide

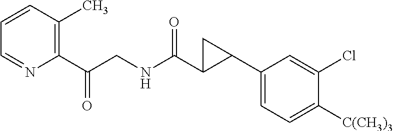

### 7A) 4-Tert-Butyl-3-Chlorophenyl Trifluoromethanesulfonate

To a pyridine (8 ml) and CH₂Cl₂ (12 ml) solution of 4-tert-butyl-3-chlorophenol (1.0 g, 5.4 mmol), trifluoromethane sulfonic acid anhydride (2.29 g, 8.1 mmol) and N,N-dimethylaminopyridine (DMAP) (12 mg, 0.1 mmol) were added and the mixture was stirred for 4 hours at 0° C. After being quenched with H₂O, the crude product was extracted with hexane, the solvent was evaporated, and the product was purified by silica gel column chromatography, eluting with gradually from hexane only to hexane/ethyl acetate (9:1), to give 4-tert-butyl-3-chlorophenyl trifluoromethanesulfonate (1.7 g, 98%) as a colorless oil. ¹H NMR (CDCl₃) δ 1.48 (9H, s), 7.13 (1H, dd, J=2.7, 8.1 Hz), 7.29 (1H, d, 2.7 Hz), 7.50 (1H, d, J=8.1 Hz)

### 7B) 1-Tert-Butyl-2-Chloro-4-Vinylbenzene

To a N,N-dimethylformamide (DMF) (50 ml) solution of 4-tert-butyl-3-chlorophenyl trifluoromethanesulfonate (Example 7A, 1.7 g, 5.3 mmol), vinyltributylstannane (1.85 g, 5.83 mmol), LiCl (2.25 g, 53 mmol) and Pd(PPh₃)₂Cl₂ (0.186 g, 0.26 mmol) were added and the mixture was stirred for 30 minutes at room temperature, followed by additional stirring for 20 hours at 30° C. The reaction was quenched with H₂O and the product was extracted with hexane. Then, evaporation of the solvent and purification by silica gel column chromatography, eluting with hexane, gave 1-tert-butyl-2-chloro-4-vinylbenzene (0.767 g, 74%) as a colorless oil.

¹H NMR (CDCl₃) δ 1.47 (9H, s), 5.26 (1H, d, J=10.8 Hz), 5.73 (1H, d, J=16.2 Hz), 6.62 (1H, J=10.8, 16.2 Hz), 7.22 (1H, d, J=8.1 Hz), 7.38 (1H, d, 8.1 Hz), 7.39 (1H, s).

### 7C) Ethyl 2-(4-Tert-Butyl-3-Chlorophenyl)Cyclopropanecarboxylate

To a toluene (5 ml) solution of 1-tert-butyl-2-chloro-4-vinylbenzene (Example 7B, 0.767 g, 3.9 mmol), 5,10,15,20-tetraphenyl-21H,23H-porphine Co(II) (Co(TPP)) (0.079 g, 0.12 mmol) and 1-methyl-1H-imidazole (0.961 g, 11.7 mmol), ethyldiazoacetate (0.623 g, 5.46 mmol) was added and the mixture was stirred for 5 minutes at room temperature, followed by additional stirring for 1 hour at 80° C. Then, evaporation of the solvent, and purification by silica gel column chromatography, eluting with gradually from hexane to hexane/ethyl acetate (10:1), gave ethyl 2-(4-tert-butyl-3-chlorophenyl)cyclopropanecarboxylate (0.97 g, 88%) as a colorless oil.

¹H NMR (CDCl₃) δ 1.24-1.33 (1H, m), 1.28 (3H, t, J=8.1 Hz), 1.45 (9H, s), 1.53-1.62 (1H, m), 1.83-1.91 (1H, m), 2.40-2.49 (1H, m), 4.17 (32H, q, J=8.1 Hz), 6.93 (1H, dd, J=2.7, 8.1 Hz), 7.08 (1H, d, J=2.7 Hz), 7.32 (1H, d, J=8.1 Hz).

MS (ESI) m/z 281 (M+H)⁺.

### 7D) 2-(4-Tert-Butyl-3-Chlorophenyl)Cyclopropanecarboxylic Acid

To a tetrahydrofuran (THF) (3 ml) solution of ethyl 2-(4-tert-butyl-3-chlorophenyl)cyclopropanecarboxylate (Example 7C, 0.97 g, 3.4 mmol), 2N NaOH (6 ml) and methanol (3 ml) were added and the mixture was stirred for 30 minutes at 80° C. After the reaction was completed, the basic mixture was acidified with 2N HCl and the product was extracted with ethyl acetate followed by evaporation of the solvent to give 2-(4-tert-butyl-3-chlorophenyl)cyclopropanecarboxylic acid (0.789 g, 92%) as a colorless oil.

MS (ESI) m/z 251 (M−H)⁻.

### 7E) 2-(4-Tert-Butyl-3-Chlorophenyl)-N-[2-(3-Methylpyridin-2-yl)-2-Oxoethyl]Cyclopropanecarboxamide

The procedure as described in example 4F was performed using 2-(4-tert-butyl-3-chloro-phenyl)cyclopropane carboxylic acid (Example 7D, 126 mg, 0.50 mmol) as starting material to give 2-(4-tert-butyl-3-chlorophenyl)-N-[2-(3-methylpyridine-2-yl)-2-oxoethyl]cyclopropanecarboxamide (55 mg, 29%) as a white solid.

¹H-NMR (CDCl₃) δ1.22-1.29 (m, 1H), 1.46 (9H, s), 1.61-1.80 (2H, m), 2.44-2.53, 2.51 (1H, m), 2.62 (3H, s), 5.00 (2H, t, J=2.7 Hz), 6.60 (1H, br), 6.96 (1H, d, J=8.1 Hz), 7.08 (1H, J=br), 7.33 (1H, d, J=8.1 Hz), 7.38 (1H, dd, J=2.7, 8.1 Hz), 7.62 (1H, d, J=8.1 Hz), 8.53 (1H, d, J=2.7 Hz).

MS (ESI) m/z 385 (M+H)⁺.

### Example 8

### 2-(4-Tert-Butyl-3-Fluorophenyl)-N-[2-(3-Trifluoromethylpyridin-2-yl)-2-Oxoethyl]Cyclopropanecarboxamide

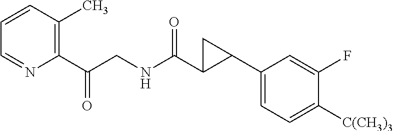

### 8A) Tert-Butyl {2-Oxo-2-[3-(Trifluoromethyl)Pyridine-2-yl]Ethyl}Carbamate

To a toluene (2 ml) solution of 2-bromo-3-trifluoromethylpyridine (0.848 g, 3.75 mmol), 1.6M hexane solution of n-BuLi was added at −78° C. and the reaction was stirred for 30 minutes. Then, a toluene (2 ml) solution of N-(tert-butoxycarbonyl)glycine N′-methoxy-N′-methylamide (0.34 g, 1.56 mmol) was added at −78° C. and the reaction stirred for 2 hours. After quenching with saturated NaHCO₃ aqueous solution, the crude product was extracted with ethyl acetate and dried over Na₂SO₄. Then, filtration and purification by silica gel column chromatography, eluting with ethyl acetate/hexane (1:4), gave tert-butyl {2-oxo-2-[3-(trifluoromethyl)pyridine-2-yl]ethyl}carbamate (0.166 g, 35%) as a yellow solid.

¹H-NMR (CDCl₃) δ 1.46 (9H, s), 4.80 (2H, d, J=4.4 Hz), 5.35 (1H, br), 7.63 (1H, dd, J=4.4, 8.1 Hz), 8.15 (1H, d, J=8.1 Hz), 8.83 (1H, d, J=4.5 Hz).

### 8B) 2-Amino-1-(3-Trifluoromethyl-2-Pyridinyl)Ethanone Dihydrochloride

To a solution of tert-butyl {2-oxo-2-[3-(trifluoromethyl)pyridine-2-yl]ethyl}carbamate (Example 8A, 0.09 g, 0.3 mmol), 10% HCl methanol (4 ml) was added and the mixture was stirred at 50° C. for 2 hours. The mixture was concentrated and dried under reduced pressure. The resulting 2-amino-1-(3-trifluoromethyl-2-pyridinyl)ethanone dihydrochloride (a white solid) was used in the next reaction without further purification.

¹H-NMR (DMSO-d₆) δ 4.66 (2H, q, J=5.6 Hz), 7.97 (1H, dd, J=4.6, 7.9 Hz), 8.48 (1H, d, J=8.0 Hz), 9.02 (1H, d, J=4.6 Hz),

MS (ESI) m/z 205 (M+H)⁺.

### 8C) 2-(4-Tert-Butyl-3-Fluorophenyl)-N-[2-(3-Trifluoromethylpyridin-2-yl)-2-Oxoethyl]Cyclopropanecarboxamide

To a CH₂Cl₂ (10 ml) solution of 2-(4-tert-butyl-3-fluorophenyl)cyclopropanecarboxylic acid (75 mg, 0.317 mmol), oxalyl dichloride (83 μl, 0.952 mmol) and N,N-dimethylformamide (DMF) (1 drop) were added and the mixture was stirred for 30 minutes at room temperature. After evaporation of the solvent, the crude residue was dried under reduced pressure. Then, to a CH₂Cl₂ (2 ml) solution of 2-amino-1-(3-trifluoromethyl-2-pyridinyl)ethanone dihydrochloride (Example 8B, 885 mg, 0.317 mmol) and diisopropylethylamine (220 μl, 1.27 mmol) was added a CH₂CH₂ (2 ml) solution of the crude residue described above and the mixture was stirred for 2 hours at room temperature. The reaction was quenched with saturated NaHCO₃ aqueous solution and the product was extracted with ethyl acetate, and dried over Na₂SO₄. Then, filtration, evaporation of the solvent, and purification by silica gel column chromatography, eluting with hexane/ethyl acetate (1:1), gave 2-(4-tert-butyl-3-fluorophenyl-N-[2-(3-trifluoromethylpyridin-2-yl)-2-oxoethylcyclopropanecarboxamide (74 mg, 55%) as a white solid.

¹H-NMR (CDCl₃) δ 1.26-1.35 (1H, m), 1.36 (9H, s), 1.55-1.76 (2H, m), 2.46-2.53 (1H, m), 4.96-4.99 (2H, dd, J=3.3, 4.6 Hz), 6.53 (1H, br), 6.72-6.85 (2H, m), 7.26 (1H, t, J=8.5 Hz), 7.65 (1H, dd, J=4.6, 7.9 Hz), 8.17 (1H, d, J=7.3 Hz), 8.85 (1H, d, J=4.5 Hz).

MS (ESI) m/z 423 (M+H)⁺.

### Example 9

### 3-(4-Tert-Butylphenyl)-2,2-Difluoro-N-[2-(3-Methylpyridin-2-yl)-2-Oxoethyl]Cyclopropanecarboxamide

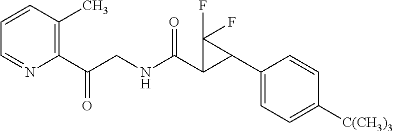

### 9A) [3-(4-Tert-Butylphenyl)-2,2-Difluorocyclopropyl]METHYL ACETATE

To a 2-methoxyethyl ether (10 ml) solution of 3-(4-tert-butylphenyl)prop-2-en-1-yl acetate (625 mg, 2.69 mmol) was added CIF₂COONa (2.05 g, 13.45 mmol) and the mixture was stirred for 30 minutes at 190° C. followed by addition of additional CIF₂COONa (0.82 g, 5.38 mmol). After additional stirring for 15 minutes, the mixture was cooled to room temperature and quenched with ethyl acetate and water. The product was extracted with ethyl acetate, dried over Na₂SO₄, filtered and the solvent evaporated. The crude product was purified by silica gel column chromatography, eluting with ethyl acetate/hexane (1:10), to give [3-(4-tert-butylphenyl)-2,2-difluorocyclopropyl]methyl acetate (801 mg, quant).

¹H-NMR (CDCl₃) δ 1.31 (9H, s), 2.10 (3H, s), 2.22-2.28 (1H, m), 2.61 (1H, dd, J=7.3, 14.7 Hz), 4.21-4.38 (2H, m), 7.16 (2H, d, J=8.1 Hz), 7.37 (2H, d, J=8.1 Hz).

### 9B) 1-[3-(4-Tert-Butylphenyl)-2,2-Difluorocyclopropyl]Methanol

To a methanol (25 ml) solution of [3-(4-tert-butylphenyl)-2,2-difluorocyclopropyl]methyl acetate (Example 9A, 801 mg, 2.69 mmol), 2N NaOH (5.0 ml) was added and the mixture was stirred for 45 minutes at room temperature. Then, the reaction was quenched with 2N HCl and the product was extracted with ethyl acetate. Drying over Na₂SO₄, filtration, evaporation, and purification by silica gel column chromatography, eluting with ethyl acetate/hexane (1:10), gave 1-[3-(4-tert-butylphenyl)-2,2-difluorocyclopropyl]methanol (365 mg, 57%).

¹H-NMR (CDCl₃) δ 1.31 (9H, s), 2.16-2.25 (1H, m), 2.54-2.63 (1H, m), 3.83-3.89 (2H, m), 7.17 (2H, d, J=7.9 Hz), 7.37 (2H, d, J=7.9 Hz).

### 9C) 3-(4-Tert-Butylphenyl)-2,2-Difluorocyclopropanecarboxylic Acid

To a benzene (15 ml) and water (20 ml) solution of 143-(4-tert-butylphenyl)-2,2-difluorocyclopropyl)-methanol (Example 9B, 120 mg, 0.5 mmol), KmnO₄ (237 mg, 1.5 mmol) and nBu₄NBr (26 mg, 0.08 mmol) were added and the mixture was stirred for 15 hours at room temperature. Then the reaction was quenched with 2N HCl and ethyl acetate and the product was extracted with ethyl acetate and dried over Na₂SO₄. After filtration and evaporation, the crude residue was used in the next reaction without purification (120.5 mg, crude).

¹H-NMR (CDCl₃) δ 1.31 (9H, s), 2.7-2.77 (1H, m), 3.42-3.55 (1H, m), 7.20 (2H, d, J=8.1 Hz), 7.39 (2H, d, J=8.1 Hz).

MS (ESI) m/z 253 (M−H)⁻.

### 9D) 3-(4-Tert-Butylphenyl)-2,2-Difluoro-N-[2-(3-Methylpyridin-2-yl)-2-Oxoethyl]Cyclopropanecarboxamide

The procedure as described in example 4F was performed using 3-(4-tert-butylphenyl)-2,2-difluorocyclopropanecarboxylic acid (Example 9C, 118 mg, 0.46 mmol) as starting material to give 3-(4-tert-butylphenyl)-2,2-difluoro-N-[2-(3-methylpyridine-2-yl)-2-oxoethyl]cyclopropanecarboxamide (31 mg, 18%) as a white solid.

¹H-NMR (CDCl₃) δ 1.31 (9H, s), 2.63 (3H, s), 2.63-2.70 (1H, m), 3.53-3.61 (1H, m), 5.06 (2 h, d, J=4.4 Hz), 6.79 (1H, br), 7.21 (2H, d, J=8.1 Hz), 7.38 (2H, d, J=8.1 Hz), 7.37-7.42 (1H, m), 7.63 (1H, d, J=7.3 Hz), 8.54 (1H, d, J=3.7 Hz).

MS (ESI) m/z 387 (M+H)⁺.

### Example 10

### 2-(4-Tert-Butyl-3-Fluorophenyl)-N-[2-(1-Methyl-1H-Imidazol-2-yl)-2-Oxoethyl]Cyclopropanecarboxamide

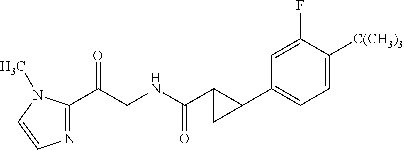

### 10A) Tert-Butyl [2-(1-Methyl-1H-Imidazol-2-yl)-2-Oxoethyl]Carbamate

To a tetrahydrofuran (20 ml) solution of 1-methylimidazole (328 mg, 4 mmol) was added n-butyllithium (2.53 ml of a 1.58M hexane sol., 4 mmol) at −78° C. over 10 minutes. After the mixture was stirred at −78° C. for 1 hour, a solution of tert-butyl {2-[methoxy(methyl)amino]-2-oxoethyl}carbamate (218 mg, 1 mmol) in THF (2 ml) was added dropwise to the reaction mixture at −78° C. and the mixture was stirred for 3 hours. Then the reaction was partitioned with saturated sodium bicarbonate aqueous solution and ethylacetate and the organic layer was separated, and dried over sodium sulfate. Then filtration and evaporation of the solvent under reduced pressure gave the crude residue which was purified by silica-gel column chromatography, eluting with hexane/ethyl acetate (1:1 to 3:1), to furnish tert-butyl [2-(1-methyl-1H-imidazol-2-yl)-2-oxoethyl]carbamate (215 mg, 90% yield) as a white solid.

¹H-NMR (300 MHz, CDCl₃) δ 1.47 (9H, s), 4.01 (3H, s), 4.72 (2H, d, J=5.5 Hz), 5.22 (1H, br s), 7.06 (1H, s), 7.16 (1H, s)⁺

MS (ESI) m/z 240 (M+H)⁺

### 10B) 2-Amino-1-(1-Methyl-1H-Imidazol-2-yl)-Ethanone Dihydrochloride

A mixture of tert-butyl [2-(1-methyl-1H-imidazol-2-yl)-2-oxoethyl]carbamate (108 mg, 0.45 mmol) and 10% hydrochloride methanol solution (2 ml) was stirred at room temperature for 16 hours. The mixture was evaporated and crystallized from ethyl acetate to furnish 2-amino-1-(1-methyl-1H-imidazol-2-yl)ethanone dihydrochloride (95 mg, quant.) as a white solid.

¹H-NMR (300 MHz, DMSO-d₆) δ 3.97 (3H, s), 4.40-4.45 (2H, m), 7.26 (1H, s), 7.69 (1H, s), 8.43 (2H, brs).

MS (ESI) m/z 140 (M+H)⁴

### 10C) 2-(4-Tert-Butyl-3-Fluorophenyl)-N-[2-(1-Methyl-1H-Imidazol-2-yl)-2-Oxoethyl]Cyclopropanecarboxamide

To a stirred solution of 2-(4-tert-butyl-3-fluorophenyl)cyclopropanecarboxylic acid (99 mg, 0.418 mmol) in dichloromethane (3 ml) was added oxalyl chloride (159 mg, 1.25 mmol) and N,N-dimethylformamide (1 drop) at 0° C. After being stirred for 1 hour at room temperature, the mixture was evaporated in vacuo and the residue was dissolved in dichloromethane (1 ml). The above solution was added to a solution of 2-amino-1-(1-methyl-1H-imidazol-2-yl)ethanone dihydrochloride (Example 10B, 89 mg, 0.418 mmol) and triethylamine (169 mg, 1.67 mmol) in dichloromethane (5 ml) at 0° C. After being stirred for 2 hours at room temperature, the mixture was diluted with dichloromethane and washed with saturated sodium bicarbonate. The organic layer was dried over sodium sulfate and concentrated in vacuo to give the crude product which was purified by silica gel column chromatography, eluting with ethyl acetate/hexane (1:1), to furnish 2-(4-tert-butyl-3-fluorophenyl)-N-[2-(1-methyl-1H-imidazol-2-yl)-2-oxoethyl]cyclopropanecarboxamide (112 mg, 75% yield) as a white solid.

¹H-NMR (300 MHz, CDCl₆) δ 1.22-1.48 (1H, m), 1.35 (9H, s), 1.56-1.78 (2H, m), 2.46-2.53 (1H, m), 4.02 (3H, s), 4.85-4.92 (2H, m), 6.50 (1H, brs), 6.70-6.72 (1H, m), 6.82-6.87 (1H, m), 7.08 (1H, s), 7.17-7.25 (2H, m)

MS (ESI) m/z 358 (M+H)⁺

### Example 11

### 2-Methyl-N-[2-(1-Methyl-1H-Imidazol-2-yl)-2-Oxoethyl]-2-[4-(2,2,2-Trifluoro-1,1-Dimethylethyl)Phenyl]Cyclopropanecarboxamide

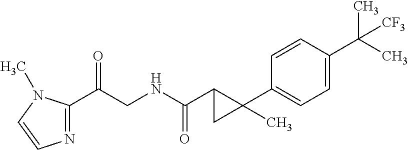

To a stirred solution of 2-methyl-2-[4-(2,2,2-trifluoro-1,1-dimethylethyl)phenyl]cyclopropanecarboxylic acid (86 mg, 0.3 mmol) in dichloromethane (2 ml) was added oxalyl chloride (114 mg, 0.9 mmol) and N,N-dimethylformamide (1 drop) at 0° C. After being stirred for 1 hour at room temperature, the mixture was evaporated in vacuo and the residue was dissolved in dichloromethane (1 ml). The above solution was added to a solution of 2-amino-1-(1-methyl-1H-imidazol-2-yl)ethanone dihydrochloride (64 mg, 0.3 mmol) and triethylamine (152 mg, 1.5 mmol) in dichloromethane (2 ml) at 0° C. After being stirred for 1 hour at room temperature, the mixture was diluted with dichloromethane and washed with saturated sodium bicarbonate. The organic layer was dried over sodium sulfate and concentrated in vacuo to give the crude product which was purified by silica gel column chromatography, eluting with ethyl acetate/hexane (1:1), to furnish 2-methyl-N-[2-(1-methyl-1H-imidazol-2-yl)-2-oxoethyl]-2-[4-(2,2,2-trifluoro-1,1-dimethylethyl)phenyl]cyclopropanecarboxamide (56 mg, 46% yield) as a white solid.

¹H-NMR (300 MHz, CDCl₆) δ 1.42 (1H, dd, J=5.1, 8.8 Hz), 1.54-1.60 (10H, m), 1.85 (1H, dd, J=5.8, 8.8 Hz), 4.02 (3H, s), 4.90 (2H, d, J=5.1 Hz), 6.44 (1H, brs), 7.08-7.30 (4H, m), 7.44 (2H, d, J=8.1 Hz) MS (ESI) m/z 408 (M+

### Example 12

### N-[2-(1-Ethyl-1H-Imidazol-2-yl)-2-Oxoethyl]-2-Methyl-2-[4-(2,2,2-Trifluoro-1,1-Dimethylethyl)Phenyl]Cyclopropanecarboxamide

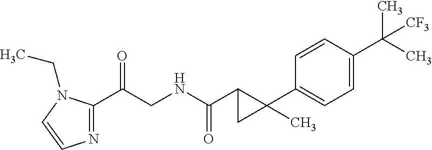

### 12A) Tert-Butyl [2-(1-Ethyl-1H-Imidazol-2-yl)-2-Oxoethyl]Carbamate

The procedure described in Example 10A was followed using 1-ethylimidazole (385 mg, 4 mmol) as starting material to furnish tert-butyl [2-(1-ethyl-1H-imidazol-2-yl)-2-oxoethyl]carbamate (224 mg, 88% yield) as a white solid.

### 12B) 2-Amino-1-(1-Ethyl-1H-Imidazol-2-yl)-Ethanone Dihydrochloride

A mixture of tert-butyl [2-(1-ethyl-1H-imidazol-2-yl)-2-oxoethyl]carbamate (76 mg, 0.3 mmol) and 10% hydrochloride methanol solution (2 ml) was treated according to the procedure described in Example 10B to furnish 2-amino-1-(1-ethyl-1H-imidazol-2-yl)ethanone dihydrochloride (68 mg, quant.) as a white solid.

MS (ESI) m/z 154 (M+H)⁺

### 12C) N-[2-(1-Ethyl-1H-Imidazol-2-yl)-2-Oxoethyl]-2-Methyl-2-[4-(2,2,2-Trifluoro-1,1-Dimethylethyl)Phenyl]Cyclopropanecarboxamide

To a stirred solution of 2-methyl-2-[4-(2,2,2-trifluoro-1,1-dimethylethyl)phenyl]cyclopropanecarboxylic acid (86 mg, 0.3 mmol) in dichloromethane (2 ml) was added oxalyl chloride (114 mg, 0.9 mmol) and N,N-dimethylformamide (1 drop) at 0° C. After being stirred for 1 hour at room temperature, the mixture was evaporated in vacuo and the residue was dissolved in dichloromethane (1 ml). The above solution was added to a solution of 2-amino-1-(1-ethyl-1H-imidazol-2-yl)ethanone dihydrochloride (Example 12B, 68 mg, 0.3 mmol) and triethylamine (152 mg, 1.5 mmol) in dichloromethane (2 ml) at 0° C. After being stirred for 1 hour at room temperature, the mixture was diluted with dichloromethane and washed with saturated sodium bicarbonate. The organic layer was dried over sodium sulfate and concentrated in vacuo to give the crude product which was purified by silica gel column chromatography, eluting with ethyl acetate/hexane (1:1), to furnish N-[2-(1-ethyl-1H-imidazol-2-yl)-2-oxoethyl]-2-methyl-2-[4-(2,2,2-trifluoro-1,1-dimethylethyl)phenyl]cyclopropanecarboxamide (70 mg, 55% yield) as a white solid.

¹H-NMR (300 MHz, CDCl₆) δ 1.39-1.46 (4H, m), 1.52-1.60 (10H, m), 1.85 (1H, dd, J=5.1, 8.1 Hz), 4.44 (2H, q, J=7.3 Hz), 4.91 (2H, d, J=4.4 Hz), 6.46 (1H, brs), 7.14-7.30 (4H, m), 7.44 (2H, d, J=8.1 Hz)

MS (ESI) m/z 422 (M+

### Example 13

### 2-[3,5-Difluoro-4-(2,2,2-Trifluoro-1,1-Dimethylethyl)Phenyl]-N-[2-(1-Ethyl-1H-Imidazol-2-yl)-2-Oxoethyl]Cyclopropanecarboxamide

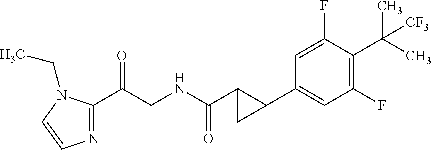

### 13A) 2-(2,6-Difluoro-4-Methoxyphenyl)-1,1,1-Trifluoropropan-2-ol

To a tetrahydrofuran (100 ml) solution of 1,3-difluoro-5-methoxybenzene (7 g, 48.6 mmol) was added 1.6 M hexane solution of n-butyllithium (30 ml, 48.6 mmol) dropwise at −78° C. over 30 minutes and the mixture was stirred for 2 hours at −78° C. 1,1,1-Trifluoroacetone (6.5 g, 58.3 mmol) was added at −78° C. and the mixture was stirred for 2 hours at −78° C. followed by additional stirring for 1 hour at room temperature. Then, the reaction was quenched with water and the product was extracted with ethyl acetate which was dried over sodium sulfate. Then, filtration, evaporation of the solvent and purification by silica gel column chromatography, eluting with hexane/ethyl acetate (10:1), furnished 2-(2,6-difluoro-4-methoxyphenyl)-1,1,1-trifluoropropan-2-ol (9.7 g, 78% yield) as a colorless oil.

¹H NMR (270 MHz, CDCl₃) δ 1.83-1.85 (3H, m), 3.94 (3H, s), 6.17 (1H, s), 6.49-6.60 (2H, m)

### 13B) 2-(1-Chloro-2,2,2-Trifluoro-1-Methylethyl)-1,3-Difluoro-5-Methoxybenzene

A thionyl chloride (25 ml) solution of 2-(2,6-difluoro-4-methoxyphenyl)-1,1,1-trifluoropropan-2-ol (Example 13A, 8.7 g, 34.1 mmol) and pyridine (26 mg, 0.34 mmol) was stirred at 70° C. for 3 hours. Then, the reaction was concentrated in vacuo and quenched with water. The product was extracted with hexane which was dried over sodium sulfate, filtration and evaporation to furnish 2-(1-chloro-2,2,2-trifluoro-1-methylethyl)-1,3-difluoro-5-methoxybenzene (8.84 g, 94% yield) as a colorless oil.

¹H NMR (270 MHz, CDCl₃) δ 2.24-2.29 (3H, m), 3.81 (3H, s), 6.44-6.54 (2H, m)

### 13C) 1,3-Difluoro-5-Methoxy-2-(2,2,2-Trifluoro-1,1-Dimethylethyl)Benzene

To a cyclohexane (100 ml) solution of 2-(1-chloro-2,2,2-trifluoro-1-methylethyl)-1,3-difluoro-5-methoxybenzene (Example 13B, 8.84 g, 32.2 mmol) was added 1.0 M hexane solution of trimethylaluminum (129 ml, 129 mmol) at room temperature and the mixture was stirred at reflux for 4 hours. Then, the reaction was quenched with 2 N-hydrochloride aqueous solution and the product was extracted with hexane and dried over sodium sulfate. Filtration and evaporation of the solvent furnished 1,3-difluoro-5-methoxy-2-(2,2,2-trifluoro-1,1-dimethylethyl)benzene (7.9 g, 97% yield) as a colorless oil.

¹H NMR (300 MHz, CDCl₃) δ 1.71 (6H, s), 3.78 (3H, s), 6.39-6.49 (2H, m)

### 13D) 3,5-Difluoro-4-(2,2,2-Trifluoro-1,1-Dimethylethyl)Phenol

A mixture of 1,3-difluoro-5-methoxy-2-(2,2,2-trifluoro-1,1-dimethylethyl)benzene (7.93 g, 31.2 mmol) and a 1 M dichloromethane solution of boron tribromide (150 ml, 150 mmol) was stirred at room temperature for 16 hours. Then, the reaction was quenched with water and the product was extracted with ethyl acetate which was dried over sodium sulfate. Then, filtration, evaporation of the solvent and purification by silica gel column chromatography, eluting with hexane/ethyl acetate (10:1), furnished 3,5-difluoro-4-(2,2,2-trifluoro-1,1-dimethylethyl)phenol (7.79 g, quant.) as a brown solid.

¹H NMR (270 MHz, CDCl₃) δ 1.71 (6H, s), 5.27 (1H, brs), 6.36-6.50 (2H, m)

### 13E) 3,5-Difluoro-4-(2,2,2-Trifluoro-1,1-Dimethylethyl)Phenyl Trifluoromethanesulfonate

To a pyridine (5 ml) and dichloromethane (10 ml) solution of 3,5-difluoro-4-(2,2,2-trifluoro-1,1-dimethylethyl)phenol (Example 13D, 456 mg, 1.9 mmol) and 4-(dimethylamino)pyridine (2 mg, 0.02 mmol) was added trifluoromethane sulfonic acid anhydride (643 mg, 2.28 mmol) dropwise at 0° C. and the mixture was stirred at 0° C. for 3 hours. Then the mixture was diluted with ethyl acetate and washed with 2M hydrochloride aqueous solution. The organic layer was dried over sodium sulfate and concentrated in vacuo to give the crude product which was purified by silica gel column chromatography, eluting with ethyl acetate/hexane (1:10), to furnish 3,5-difluoro-4-(2,2,2-trifluoro-1,1-dimethylethyl)phenyl trifluoromethanesulfonate (440 mg, 62% yield) as a colorless oil.

¹H NMR (300 MHz, CDCl₃) δ 1.75-1.77 (6H, m), 6.86-6.95 (2H, m)

### 13F) 5-Ethenyl-1,3-Difluoro-2-(2,2,2-Trifluoro-1,1-Dimethylethyl)Benzene

To a N,N-dimethylformamide (DMF) (5 ml) solution of 3,5-difluoro-4-(2,2,2-trifluoro-1,1-dimethylethyl)phenyl trifluoromethanesulfonate (440 mg, 1.18 mmol), vinyltributylstannane (450 mg, 1.42 mmol), lithium chloride (500 mg, 11.8 mmol) and Pd(PPh₃)₂Cl₂ (41 mg, 0.059 mmol) were added and the mixture was stirred for 2 hours at 70° C. The reaction was quenched with water and the product was extracted with hexane. Then, evaporation of the solvent and purification by silica gel column chromatography, eluting with hexane, gave the crude product of 5-ethenyl-1,3-difluoro-2-(2,2,2-trifluoro-1,1-dimethylethyl)benzene including vinyltributylstannane (crude 829 mg) as a colorless oil.

¹H NMR (270 MHz, CDCl₃) δ 5.66 (1H, d, J=10.6 Hz), 6.05 (1H, d, J=17.8 Hz), 6.86 (1H, dd, J=10.6, 17.8 Hz), 7.14-7.22 (2H, m)

### 13G) ETHYL 2-[3,5-Difluoro-4-(2,2,2-Trifluoro-1,1-Dimethylethyl)Phenyl]Cyclopropanecarboxylate

To a toluene (3 ml) solution of crude 5-ethenyl-1,3-difluoro-2-(2,2,2-trifluoro-1,1-dimethylethyl)benzene (Example 13F, 829 mg), 5, 10, 15, 20 tetraphenyl-21H, 23H porphine Co(II) (Co(TPP)) (24 mg, 0.035 mmol) and 1-methyl-1H-imidazole (484 mg, 5.9 mmol) were added ethyl diazoacetate (262 mg, 2.6 mmol) and the mixture was stirred at 80° C. for 1 hour. Then the reaction was concentrated and the crude residue was applied to a silica gel chromatography column and eluted with gradually from hexane to hexane/ethylacetate (10:1) to furnish crude ethyl 2-[3,5-difluoro-4-(2,2,2-trifluoro-1,1-dimethylethyl)phenyl]cyclopropanecarboxylate including vinyltributylstannane as a black oil.

¹H NMR (270 MHz, CDCl₃) δ 0.88-1.93 (12H, m), 2.40-2.47 (1H, m), 4.14-4.20 (2H, m), 6.57-6.66 (2H, m) 13H) 2-[3,5-Difluoro-4-(2,2,2-Trifluoro-1,1-Dimethylethyl)Phenyl]Cyclopropanecarboxylic Acid

To a tetrahydrofuran (THF) (5 ml) solution of crude ethyl 2-[3,5-difluoro-4-(2,2,2-trifluoro-1,1-dimethylethyl)phenyl]cyclopropanecarboxylate including vinyltributylstannane, 2M sodium hydroxide aqueous solution (2 ml) and methanol (5 ml) were added and the mixture stirred at room temperature for 6 hours. After the reaction was complete, the basic mixture was washed with diethyl ether, the separated aqueous layer was acidfied with 2M hydrochloride aqueous solution and the product was extracted with ethylacetate followed by evaporation of the solvent to furnish 2-[3,5-difluoro-4-(2,2,2-trifluoro-1,1-dimethylethyl)phenyl]cyclopropanecarboxylic acid (198 mg, 54% yield in 3 steps) as a white solid.

MS (ESI) m/z 307 (M−H)⁺.

### 13I) 2-[3,5-Difluoro-4-(2,2,2-Trifluoro-1,1-Dimethylethyl)Phenyl]-N-[2-(1-Ethyl-1H-Imidazol-2-yl)-2-Oxoethyl]Cyclopropanecarboxamide

To a N,N-dimethylformamide (DMF) (0.5 ml) solution of 2-[3,5-difluoro-4-(2,2,2-trifluoro-1,1-dimethylethyl)phenyl]cyclopropanecarboxylic acid (Example 13H, 61 mg, 0.2 mmol), N,N,N′,N′-tetramethyl-O-(1H-benzotriazol-1-yl)uronium hexafluorophosphate (HBTU) (90 mg, 2.4 mmol), triethylamine (0.14 ml, 1.0 mmol) and 2-amino-1-(1-ethyl-1H-imidazol-2-yl)ethanone dihydrochloride (Example 12B, 31 mg, 0.2 mmol) were added and the mixture was stirred for 1 hour at room temperature. Then, the reaction was quenched with saturated sodium bicarbonate aqueous solution, and the product was extracted with ethyl acetate which was dried over sodium sulfate. Then, filtration, evaporation of the solvent and purification by silica gel column chromatography, eluting with hexane/ethyl acetate (1:1), gave 2-[3,5-difluoro-4-(2,2,2-trifluoro-1,1-dimethylethyl)phenyl]-N-[2-(1-ethyl-1H-imidazol-2-yl)-2-oxoethyl]cyclopropanecarboxamide (36 mg, 41% yield) as a white solid.

¹H-NMR (300 MHz, CDCl₆) δ 1.24-1.28 (1H, m), 1.43 (3H, t, J=7.3 Hz), 1.60-1.75 (8H, m), 2.44-2.51 (1H, m), 4.44 (2H, q, J=7.3 Hz), 4.89 (2H, d, J=5.1 Hz), 6.56 (1H, brs), 6.60-6.68 (2H, m), 7.16-7.20 (2H, m) MS (ESI) m/z 444 (M+H)⁺

All the Examples described above were tested in the human VR1 antagonist assay method described hereinabove and the results are presented in the following table:

| Example No. | hVR1 IC₅₀ (nM) |
|---|---|
| 1 | 102 |
| 2 | 1011 |
| 3 | 704 |
| 4 | 99 |
| 5 | 111 |
| 6 | 134 |
| 7 | 92 |
| 8 | 102 |
| 9 | 306 |
| 10 | 238 |
| 11 | 451 |
| 12 | 340 |
| 13 | 69 |

IC₅₀: the concentration of the individual compound required to reduce Ca²⁺ influx capsaicin-evoked by 50%.

1. A compound of formula (I): 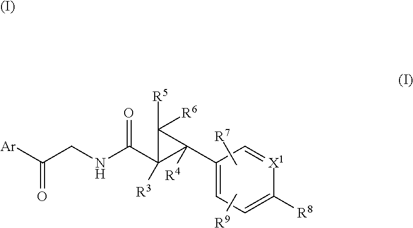
wherein Ar represents 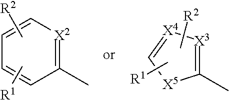
X¹ represents CH, CR⁷ or N;
X² represents CH, CR¹ or N;
X³ represents N, X⁴ represents CH or CR¹ and X⁵ represents S, NH or NR²; or X³ represents CH or CR¹,
X⁴ represents N and X⁵ represents NH or NR²;
R¹, R², R⁷ and R⁹ each independently represent hydrogen, halogen, hydroxy, (C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, halo(C₁-C₆)alkyl, (C₁-C₆)alkylthio, (C₁-C₆)alkylsulfinyl, (C₁-C₆)alkylsulfonyl, [(C₁-C₆)alkyl]NH—, [(C₁-C₆)alkyl]₂N—, H₂N—(C₁-C₆)alkoxy, (C₁-C₆)alkyl-NH—(C₁-C₆)alkoxy, [(C₁-C₆)alkyl]₂N(C₁-C₆)alkoxy; H₂N—(C₁-C₆)alkoxy-(C₁-C₆)alkyl, (C₁-C₆)alkyl-NH—(C₁-C₆)alkoxy-(C₁-C₆)alkyl, or [(C₁-C₆)alkyl]₂N(C₁-C₆)alkoxy-(C₁-C₆)alkyl;
R³, R⁴, R⁵ and R⁶ each independently represent hydrogen, halogen, (C₁-C₆)alkyl, hydroxy(C₁-C₆)alkyl or halo(C₁-C₆)alkyl; and
R⁸ represents halogen, (C₁-C₆)alkyl, halo(C₁-C₆)alkyl, (C₁-C₆)alkoxy, hydroxy(C₁-C₆)alkoxy, (C₁-C₆)alkoxy-(C₁-C₆)alkyl, (C₁-C₆)alkoxy-(C₁-C₆)alkoxy, [(C₁-C₆)alkyl]NH—, or [(C₁-C₆)alkyl]₂N—, or R⁷ and R⁸, when attached to adjacent carbon atoms, may be taken together with the carbon atoms to which they are attached to form a 5- to 8-membered cycloalkyl ring or heterocyclic ring in which one or two non-adjacent carbon atoms are optionally replaced by oxygen, sulfur or NH groups, wherein the cycloalkyl ring or the heterocyclic ring is unsubstituted or substituted with one or more substituents selected from the group consisting of hydroxy, (C₁-C₆)alkyl, (C₁-C₆)alkoxy and hydroxy(C₁-C₆)alkyl;
or a pharmaceutically acceptable salt or solvate thereof.

2. A compound according to claim 1 wherein Ar represents 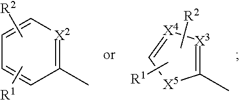
X² represents N, CH or CR¹;
X³ represents N, X⁴ represents CH, and X⁵ represents NH or NR¹ respectively.

3. A compound according to any claim 1 or claim 2, wherein X¹ represents CH or CR⁷.

4. A compound according to any one of claims 1 to 3, wherein R¹ and R² are each independently hydrogen, hydroxy, (C₁-C₆)alkyl, halo(C₁-C₆)alkyl and (C₁-C₆)alkoxy

5. A compound according to any one of claims 1 to 4, wherein R³, R⁴, R⁵ and R⁶ are each independently hydrogen, halogen or (C₁-C₆)alkyl.

6. A compound according to any one of claims 1 to 5, wherein R⁷ and R⁹ are each independently hydrogen or halogen.

7. A compound according to any one of claims 1 to 6, wherein R⁸ represents (C₁-C₆)alkyl or halo(C₁-C₆)alkyl.

8. A compound according to any one of claims 1 to 7, wherein R⁸ represents tert-butyl, trifluoromethyl or 2,2,2-trifluoro-1,1-dimethylethyl.

9. A compound according to claim 1 selected from:
2-(4-tert-butylphenyl)-N-[2-(3-methylpyridin-2-yl)-2-oxoethyl]cyclopropanecarboxamide;
2-(4-tert-butylphenyl)-N-[2-(5-methoxy-2-methylphenyl)-2-oxoethyl]cyclopropanecarboxamide;
2-(4-tert-butylphenyl)-N-[2-(5-hydroxy-2-methylphenyl)-2-oxoethyl]cyclopropanecarboxamide;
2-(4-tert-butyl-3-fluorophenyl)-N-[2-(3-methylpyridin-2-yl)-2-oxoethyl]cyclopropanecarboxamide;
N-[2-(3-methylpyridin-2-yl)-2-oxoethyl]-2-[4-(2,2,2-trifluoro-1,1-dimethylethyl)]cyclopropanecarboxamide;
2-(4-tert-butylphenyl)-2-methyl-N-[2-(3-methylpyridin-2-yl)-2-oxoethyl]cyclopropanecarboxamide;
2-(4-tert-butyl-3-chlorophenyl)-N-[2-(3-methylpyridin-2-yl)-2-oxoethyl]cyclopropanecarboxamide;
2-(4-tert-butyl-3-fluorophenyl)-N-[2-(3-trifluoromethylpyridin-2-yl)-2-oxoethyl]cyclopropanecarboxamide;
3-(4-tert-butylphenyl)-2,2-difluoro-N-[2-(3-methylpyridin-2-yl)-2-oxoethyl]cyclopropanecarboxamide;
2-(4-tert-butyl-3-fluorophenyl)-N-[2-(1-methyl-1H-imidazol-2-yl)-2-oxoethyl]cyclopropanecarboxamide;
2-methyl-N-[2-(1-methyl-1H-imidazol-2-yl)-2-oxoethyl]-2-[4-(2,2,2-trifluoro-1,1-dimethylethyl)phenyl]cyclopropanecarboxamide;
N-[2-(1-ethyl-1H-imidazol-2-yl)-2-oxoethyl]-2-methyl-2-[4-(2,2,2-trifluoro-1,1-dimethylethyl)phenyl]cyclopropanecarboxamide; and
2-[3,5-difluoro-4-(2,2,2-trifluoro-1,1-dimethylethyl)phenyl]-N-[2-(1-ethyl-1H-imidazol-2-yl)-2-oxoethyl]cyclopropanecarboxamide;
or a pharmaceutically acceptable salt or solvate thereof.

10. A pharmaceutical composition including a compound of the formula (I) or a pharmaceutically acceptable salt thereof, as defined in any one of claims 1 to 9, together with a pharmaceutically acceptable excipient.

11. A compound of formula (I), or a pharmaceutically acceptable salt or solvate thereof, as defined in any one of claims 1 to 9, for use as a medicament.

12. The use of a compound of the formula (I) or a pharmaceutically acceptable salt or composition thereof, as defined in any one of claims 1 to 9, in the manufacture of a medicament for the treatment of a disease for which a VR1 antagonist is indicated.

13. A use according to claim 12 where the disease is selected from acute cerebral ischemia, pain, chronic pain, neuropathic pain, inflammatory pain, post herpetic neuralgia, neuropathies, neuralgia, diabetic neuropathy, HIV-related neuropathy, nerve injury, rheumatoid arthritic pain, osteoarthritic pain, burns, back pain, visceral pain, cancer pain, dental pain, headache, migraine, carpal tunnel syndrome, fibromyalgia, neuritis, sciatica, pelvic hypersensitivity, pelvic pain, menstrual pain; bladder disease, such as incontinence, micturition disorder, renal colic and cystitis; inflammation, such as burns, rheumatoid arthritis and osteoarthritis; neurodegenerative disease, such as stroke, post stroke pain and multiple sclerosis; pulmonary disease, such as asthma, cough, chronic obstructive pulmonary disease (COPD) and broncho constriction; gastrointestinal, such as gastroesophageal reflux disease (GERD), dysphagia, ulcer, irritable bowel syndrome (IBS), inflammatory bowel disease (IBD), colitis and Crohn's disease; ischemia, such as cerebrovascular ischemia; emesis, such as cancer chemotherapy-induced emesis, and obesity.

14. A method for the treatment of a disease for which an VR1 antagonist is indicated in a mammal, including a human, which includes administering to said mammal a therapeutically effective amount of a compound of formula (I), or a pharmaceutically acceptable salt or solvate thereof, as defined in any one of claims 1 to 9.

15. A combination of a compound of the formula (I) or a pharmaceutically acceptable salt or solvate thereof, as defined in any one of claims 1 to 9, and another pharmacologically active agent.
